(19) United States
(12) Patent Application Publication
(10) Pub. No.: US 2026/0356794 A1
(43) Pub. Date: Nov. 12, 2026

(54) USES OF NOVEL LUMINESCENT SURFACTANT COMPOUNDS

(71) Applicant: NORTH-WEST UNIVERSITY, Potchefstroom (ZA)

(72) Inventors: Hermanus Cornelius Moolman VOSLOO, Potchefstroom (ZA); Daniel Petrus OTTO, Potchefstroom (ZA); Frans Johannes SMIT, Potchefstroom (ZA); Ettiene Hugo WIESE, Potchefstroom (ZA)

(21) Appl. No.: 19/486,587

(22) PCT Filed: May 24, 2024
(86) PCT No.: PCT/IB2024/055070

§ 371 Date: Nov. 21, 2025

(30) Foreign Application Priority Data
May 26, 2023 (ZA) 2023/05669
Feb. 27, 2024 (ZA) 2024/01655

(51) Int. Cl. C07C 229/26; A61K 49/00; C07D 265/33

(52) U.S. Cl. CPC C07C 229/26; A61K 49/0021; C07D 265/33

(57) ABSTRACT

The present invention relates to uses of novel luminescent surfactant compounds. The invention further relates to novel uses of novel types of luminescent Guerbet-type surfactant compounds.

### FIELD OF APPLICATION OF THE INVENTION

The present invention relates to uses of novel luminescent surfactant compounds. The invention further relates to novel uses of novel types of luminescent Guerbet-type surfactant compounds.

### BACKGROUND TO THE INVENTION

South African provisional patent application Number 2023/05668 and South African provisional patent application Number 2024/01654 are incorporated herein by reference.

Surfactants are molecules which possess both hydrophobic and hydrophilic groups. This characteristic allows the surfactant to bind with oil and water simultaneously, thereby lowering the surface tension of water as example. A wide range of synthetic surfactants exist today and can be applied for different purposes.

The four broad categories into which surfactants can be classified include amphoteric (or zwitterionic), anionic, cationic and non-ionic.

Zwitterionic surfactants, which contain both positively and negatively charged head groups, are interesting molecules because of their many unique properties. In general, they are mild to the skin and eyes, exhibit low toxicity, display excellent water solubility, broad isoelectric ranges, high foam stability, resistance to hard water and degradation by oxidizing agents.

Factors affecting zwitterionic surfactants to various degrees may include, but are not limited to, temperature, pH and added electrolytes.

Cationic surfactants are surfactants that have a positively charged functional group (hydrophilic head) whilst anionic surfactants are surfactants that carry a negatively charged functional group (hydrophilic head).

Guerbet alcohols are a type of compound named after the French chemist Marcel Guerbet, who first discovered them in 1899. Guerbet was investigating the reactions of alcohols with sodium metal when he discovered a new type of alcohol with a branched structure. He later found that these branched alcohols could be used as surfactants.

In the early 20th century, Guerbet alcohols were used mainly as solvents and intermediates in the synthesis of other compounds. It was not until the 1950s that their surfactant properties were fully realized, and they began to be used in a range of industrial applications. Guerbet-type surfactants became increasingly popular in the 1970s and 1980s as more industries discovered their unique properties. The branched structure and unique functional groups of Guerbet-type surfactants gives them excellent surface activity and stability, making them highly effective in reducing surface tension and stabilizing emulsions.

The popularity of Guerbet-type surfactants continued to grow in the 1990s and 2000s as advances in technology and chemistry allowed for the development of new Guerbet-type surfactant formulations with improved properties.

Due to their versatile properties, they are utilized in various industries which include but are not limited to, detergents, personal care, pharmaceuticals, agrochemicals, mining, metalworking, paper, lubricating products, latex systems, plastics, and composites.

The unique properties of Guerbet-type surfactants make them valuable and versatile in many industries, with the potential for further expansion as research and technology evolve. However, Guerbet-type surfactants have a unique chemical structure consisting of branched alkyl chains that can make them more challenging to manufacture compared to other surfactants with linear alkyl chains. This structural complexity requires specialised production processes and equipment, which can result in higher production costs and lower yields. As a result, the supply of Guerbet-type surfactants may be limited, and they may not be as widely available as other types of surfactants.

Regarding the surfactants market size, in the year 2020 the global surfactants market was valued at USD 33 billion and projected to grow at a CAGR of 3.5% between 2021-2027, reaching the USD 44 billion mark by the end of 2027. Of interest, Guerbet-type surfactants are expected to grow from USD 1.1 billion in 2021 to USD 1.4 billion in 2029 at a CAGR of 3.9%. The cosmetics and personal care segment is projected to be the main driver of the global Guerbet alcohols market during the forecast period, with the Asia Pacific being the market leader. In this period, it is projected that the anionic amino acid surfactants will lose market share to cationic and zwitterionic surfactants.

Although there is a growing trend toward the demand of bio-based surfactants, the high cost of manufacturing these surfactants has a negative impact on the forecasted growth of this market. Bio-surfactants are more expensive in comparison to traditional synthetic surfactants.

The high costs of producing bio-surfactants are driven by the high cost of raw materials and offset economies of scale of bio-surfactants. This is particularly true in the case of fast-moving consumer goods (FMCG) companies such as Reckitt Benckiser, P&G, L'Oréal, and Colgate where the raw material prices have a definite impact on the consumer goods. For these reasons, there is a huge demand for synthetic surfactants.

Quantum dots have various applications in single-electron transistors, solar cells, LEDs, lasers, single-photon sources, second-harmonic generation, quantum computing, cell biology research, microscopy, medical imaging and the like. A fundamental drawback with quantum dots is the toxicity of the materials used, such as lead and cadmium. Rare earth metals used for quantum dots are not only expensive but are also not abundant, making their use in quantum dot applications unsustainable. In addition, the organic framework surrounding quantum dots are non-biodegradable.

It is well known that luminescent techniques in the form of fluorescence microscopy are commonly used to measure and characterise surfactants. These techniques are often used to discern microstructural characteristics of micelles and hybrid materials and to afford direct visualization of polymer-surfactant interactions and DNA compaction, offering a granular view of conformational changes and the behaviour of surfactants at the molecular level. Such luminescent techniques are useful tools when characterizing surfactant properties of surfactants.

Dyes are defined as unsaturated organic compounds that selectively absorb wavelengths within the visible spectrum (doi: 10.3390/ma6020580). Amphiphilic type dyes are unsaturated organic compounds with distinct hydrophobic and hydrophilic segments, mirroring surfactant structures (Tehrani-Bagha & Holmberg, 2013). These dyes absorb selected wavelengths within the visible spectrum and demonstrate surface-active properties, making them similar in function to surfactants. Notable among these is diquat dibromide, a water-soluble dye used in various applications. Despite the widespread use of these dyes, the inherent surfactant capabilities of these dye molecules are typically insufficient, necessitating the concurrent use of a cosurfactant, often of the non-ionic variety, to enhance their performance. Despite this limitation, such compounds are consistently referred to as amphiphilic or surface-active dyes, underscoring their dual functionality.

Fluorescent surfactants derived from conventional dyes, such as Rhodamine B and Eosin Y, exhibit surfactant characteristics to a certain degree (https://doi.org/10.1515/pac-2019-0219).

These compounds exhibit CMCs ranging from 0.5 to 3 mmol/L, with surface tensions exceeding 60 mN/m, thereby indicating their suboptimal surfactant performance. This limitation is predominantly attributed to the disproportion in the amphiphilic balance, with a pronounced bias towards hydrophobicity, diminishing their surfactant efficacy.

Further, specific non-aromatic aliphatic surfactants are utilized for their ability to chelate lanthanides or encapsulate materials like CdSe quantum dots, despite lacking fluorescence themselves (Chen & Rosenzweig, 2002). These surfactants operate by chelating to lanthanides or encapsulating quantum dot materials like CdSe quantum dots, leveraging their surface-active properties for encapsulation (Chen & Rosenzweig, 2002). Nevertheless, their utility is subject to constraints, including economic considerations, owing to the high cost associated with lanthanides.

There is thus a clear need which exists for new uses of surfactant compounds in order to meet the demand for niche applications and new technological developments across the globe.

### OBJECT OF THE INVENTION

It is accordingly an object of the present invention to provide uses of new luminescent surfactant compounds, including new uses of novel types of luminescent surfactant compounds, which overcome, at least partially, the abovementioned disadvantages and limitations and/or which will provide useful alternatives to meet the demand for niche applications and new technological developments worldwide.

### SUMMARY OF THE INVENTION

It is to be appreciated that the following definitions are applicable throughout the specification.

Luminescence refers to the emission of light due to at least one of a chemical reaction, electrical energy, or subatomic motions. Fluorescence refers to a form of luminescence, wherein a substance, such as a compound, absorbs light or other electromagnetic radiation at a wavelength and emits light at a shorter wavelength after the brief excitation.

It is to be appreciated that throughout the body of the specification “luminescence” and “fluorescence” may be used interchangeably unless the context dictates otherwise.

According to a first aspect of the present invention, there is provided use of luminescent surfactant compounds, having the chemical structure:

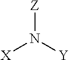

wherein:
(i) X is either COR or CH₂R, wherein R is either R′CH(CH₂)₂R′ or R′CCHCH₂R′, and wherein R′ is CₙH₂ₙ₊₁ (n=1, 2, . . . );
(ii) Y is selected from the group consisting of H; R′, X; (CH₂)ₙCO₂M (n=0, 1, 2, . . . ; M=H, R′, Li, Na, K, Rb, Cs, Fr, Er, Gd, . . . ); (CH₂)ₙA (n=0, 1, 2, . . . ; A=OH, OR′, SH, SR′, NH₂, NHR′, NR′₂, CONH₂, CONHR′, CONR′₂, aromatic groups, heteroaromatic groups, halogens (F, Cl, Br, I)); or CH₂CH(CH₃)O(CH₂CH₂O)ₙH (n=0, 1, 2, . . . ); and
(iii) Z is selected from the group consisting of H; R′; (CH₂)ₙCO₂M (n=1, 2, . . . ; M=H, R′, Li, Na, K, Rb, Cs, Fr, Er, Gd, . . . ); CH₂CH(CH₃)O(CH₂CH₂O)ₙH (n=0, 1, 2, . . . ); or (CH₂)ₙA (n=0, 1, 2, . . . ; A=OH, OR′, SH, SR′, NH₂, NHR′, NR′₂, CONH₂, CONHR′, CONR′₂, aromatic groups, heteroaromatic groups, halogens (F, Cl, Br, I)),
   in luminescence applications selected from the group consisting of quantum dots, UV-down converters, solar cells, smart materials, sensors, and bio-imaging.

According to a second aspect of the present invention, there is provided use of luminescent surfactant compounds having the chemical structure:

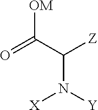

wherein:
(i) X is either COR or CH₂R, wherein R is either R′CH(CH₂)₂R′ or R′CCHCH₂R′, and wherein R′ is CₙH₂ₙ₊₁ (n=1, 2, . . . );
(ii) Y is selected from the group consisting of H; R′, X; (CH₂)ₙCO₂M (n=0, 1, 2, . . . ; M=H, R′, Li, Na, K, Rb, Cs, Fr, Er, Gd, . . . ); (CH₂)ₙA (n=0, 1, 2, . . . ; A=OH, OR′, SH, SR′, NH₂, NHR′, NR′₂, CONH₂, CONHR′, CONR′₂, aromatic groups, heteroaromatic groups, halogens (F, Cl, Br, I)); or CH₂CH(CH₃)O(CH₂CH₂O)ₙH (n=0, 1, 2, . . . ); and
(iii) Z is selected from the group consisting of H; R′; (CH₂)ₙA (n=0, 1, 2, . . . ; A=OH, OR′, SH, SR′, NH₂, NHR′, NR′₂, CONH₂, CONHR′, CONR′₂, aromatic groups (e.g. C₆H₅, C₆H₄OH), heteroaromatic groups, halogens (F, Cl, Br, I)); (CH₂)ₙCO₂M (n=1, 2, . . . ; M=H, R′, Li, Na, K, Rb, Cs, Fr, Er, Gd, . . . );

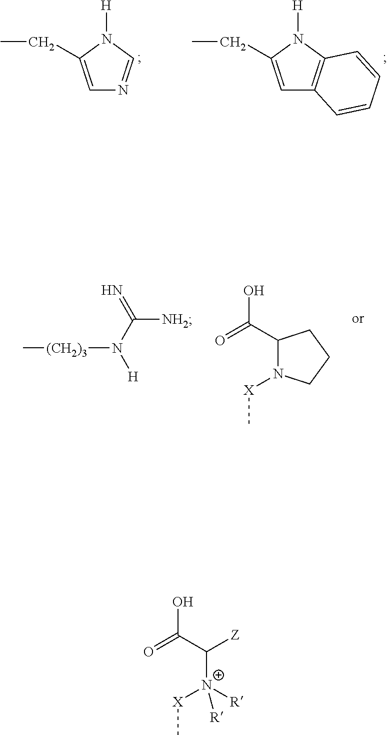

wherein A=OH (SER); SH (CYS); CH₂SMe (MET); COOH (ASP); CH₂COOH (GLU); (CH₂)₃NH₂ (LYS); CONH₂ (ASP); CH₂CONH₂ (GLN); CH(OH)Me (THR); and
wherein ALA=alanine; ASP=aspartic acid; GLU=glutamic acid; GLY=glycine; IDA=iminodiacetic acid; VAL=valine; LEU=leucine; ILE=isoleucine; PHE=phenylalanine; SER=serine; CYS=cysteine; MET=methionine; LYS=lysine; GLN=glutamine; THR=threonine,
   in luminescence applications selected from the group consisting of quantum dots, UV-down converters, solar cells, smart materials, sensors, and bio-imaging.

According to a third aspect of the present invention, there is provided for use of luminescent surfactant compounds having the chemical structure:

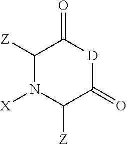

wherein:
(i) X is either COR or CH₂R, wherein R is either R′CH(CH₂)₂R′ or R′CCHCH₂R′, and wherein R′ is CₙH₂ₙ₊₁ (n=1, 2, . . . );
(ii) D is selected from the group consisting of O; N(CH₂)ₙA or N(C₆H₄)ₙA (n=0, 1, 2, . . . ; A=OH, OR′, SH, SR′, NH₂, NHR′, NR′₂, CONH₂, CONHR′, CONR′₂, aromatic groups (e.g. C₆H₅, C₆H₄OH), heteroaromatic groups or halogens (F, Cl, Br, I)); and
(iii) Z is selected from the group consisting of H; R′; N(CH₂)ₙA (n=0, 1, 2, . . . ; A=OH, OR′, SH, SR′, NH₂, NHR′, NR′₂, CONH₂, CONHR′, CONR′₂, aromatic groups (e.g. C₆H₅, C₆H₄OH), heteroaromatic groups, halogens (F, Cl, Br, I)); or (CH₂)ₙCO₂M (n=1, 2, . . . ; M=H, R′, Li, Na, K, Rb, Cs, Fr, Er, Gd, . . . ),
   in luminescence applications selected from the group consisting of quantum dots, UV-down converters, solar cells, smart materials, sensors, and bio-imaging.

According to a fourth aspect of the present invention, there is provided use of luminescent surfactant compounds having the chemical structure:

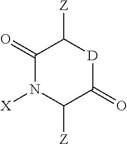

wherein:
(i) X is either COR or CH₂R, wherein R is either R′CH(CH₂)₂R′ or R′CCHCH₂R′, and wherein R′ is CₙH₂ₙ₊₁ (n=1, 2, . . . );
(ii) D is selected from the group consisting of O; N(CH₂)ₙA or N(C₆H₄)ₙA (n=0, 1, 2, . . . ; A=OH, OR′, SH, SR′, NH₂, NHR′, NR′₂, CONH₂, CONHR′, CONR′₂, aromatic groups (e.g. C₆H₅, C₆H₄OH), heteroaromatic groups or halogens (F, Cl, Br, I)); and
(iii) Z is selected from the group consisting of H; R′; N(CH₂)ₙA (n=0, 1, 2, . . . ; A=OH, OR′, SH, SR′, NH₂, NHR′, NR′₂, CONH₂, CONHR′, CONR′₂, aromatic groups (e.g. C₆H₅, C₆H₄OH), heteroaromatic groups, halogens (F, Cl, Br, I)); or (CH₂)ₙCO₂M (n=1, 2, . . . ; M=H, R′, Li, Na, K, Rb, Cs, Fr, Er, Gd, . . . ),
   in luminescence applications selected from the group consisting of quantum dots, UV-down converters, solar cells, smart materials, sensors, and bio-imaging.

According to a fifth aspect of the present invention, there is provided use of luminescent linear heteroatom functionalized surfactant compounds in luminescence applications selected from the group consisting of quantum dots, UV-down converters, solar cells, smart materials, sensors, and bio-imaging.

In terms of the invention, the luminescent linear heteroatom functionalized surfactant compounds may include both saturated and unsaturated hydrocarbons.

It will be appreciated that the linear heteroatom functionalized surfactant compounds may be any suitable commercially available surfactant. Here, the Applicant has now surprisingly and unexpectantly found that these linear heteroatom functionalized surfactant compounds demonstrate luminescence properties.

In an embodiment of the invention, the luminescent linear heteroatom functionalized surfactant compounds include, but are not limited to, the following: Rewoteric AMC 2CNM; Rewopol SBF A30B; Tego SMS 60; Varisoft 432 CG and Varisoft 300.

The above-mentioned and other characteristics, features and advantages of the present invention will become apparent from the following detailed description, taken in conjunction with the accompanying drawings which illustrate, by way of example, the principles of the invention. This description is given for the sake of example only, without limiting the scope of the invention.

### BRIEF DESCRIPTION OF THE DRAWINGS

Preferred embodiments of the invention are described below with reference to the accompanying figures, wherein:

FIG. 1 is emission spectra of bCₙIDA (n=12, 16, 18, 20, 22) surfactants of the present invention in MeOH;

FIG. 2 is emission spectra of bC₁₆ASP vs bC₁₆IDA surfactants of the present invention in MeOH;

FIG. 3 emission, in shades of blue, of three bCₙIDA (n=12, 16, 22) surfactant films of the present invention after 365 nm irradiation;

FIG. 4 is a photograph of a yellow wax of bC₂₂ASP of the present invention radiated with 365 nm to yield a bright blue wax;

FIG. 5 is a photograph of bC₂₀IDA_{Me} as a wax of the present invention emitting a green colour;

FIG. 6 is a photograph of bC₂₂ASP dissolved in DMSO and excited with sunlight and 365 nm light emitting a violet colour;

FIG. 7 illustrates a polymer sheet impregnated with a surfactant of the present invention for down conversion of UV-light;

FIG. 8 are photographs of cellulose acetate (CA) sheets impregnated with bC₁₂p-ABA, (a) sheet in visible light, (b) sheet irradiated with 365 nm light emitting a bright blue colour, (c) cut out of sheet to illustrate transparency, (d, e) shards of CA sheets emitting a bright blue colour;

FIG. 9 demonstrates the influence of the luminescent surfactant on the film contraction of a CA sheet. FIG. 9(a) CA with bC₁₂p-ABA and FIG. 9(b) blank CA;

FIG. 10 demonstrates a solar cell on a solar simulator with bC₁₂p-ABA impregnated CA film (FIG. 10(a)) and FIG. 10(b) shows the wattage as a function of Voltage, wherein the bottom line represents the solar cell without film and the top line represents the solar cell with film;

FIG. 11 shows a representation of a LSC based solar cell (FIG. 11(a)), FIG. 11(b) shows a bC₁₂p-ABA coating on a microscope slide in sunlight emitting a purple colour on the left, and a bC₁₂p-ABA coating on a microscope slide under 365 nm light emitting a brilliant blue colour on the right;

FIG. 12 shows photos demonstrating the luminescence in shades of blue of commercial linear heteroatom functionalized surfactant compounds according to the fifth aspect of the present invention where the surfactants are dissolved in ethanol (365 nm) as shown in FIG. 12(a) and where the surfactants are as formulated by EVONIK (356 nm);

FIG. 13 is a diagram showing a luminescent solar concentrator (LSC) photon transfer mechanism;

FIG. 14 are diagrams showing the applications of the surfactants of the present invention in smart windows;

FIG. 15 is a diagram of a solar panel developed by SHARP for purposes of demonstrating the use of the surfactants of the present invention in UV-downconverters; and

FIG. 16 depicts the general structure of luminescent surfactant compounds in accordance with the present invention.

Given the nature of the present invention, it will be appreciated that in order to exemplify the invention clearly and fully, colour Figures are required, and in fact essential, in order to demonstrate the luminescent properties exhibited by the surfactant compounds. These can be accessed on PatentScope and can be furnished upon request thereof.

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

### DESCRIPTION OF PREFERRED EMBODIMENTS OF THE INVENTION

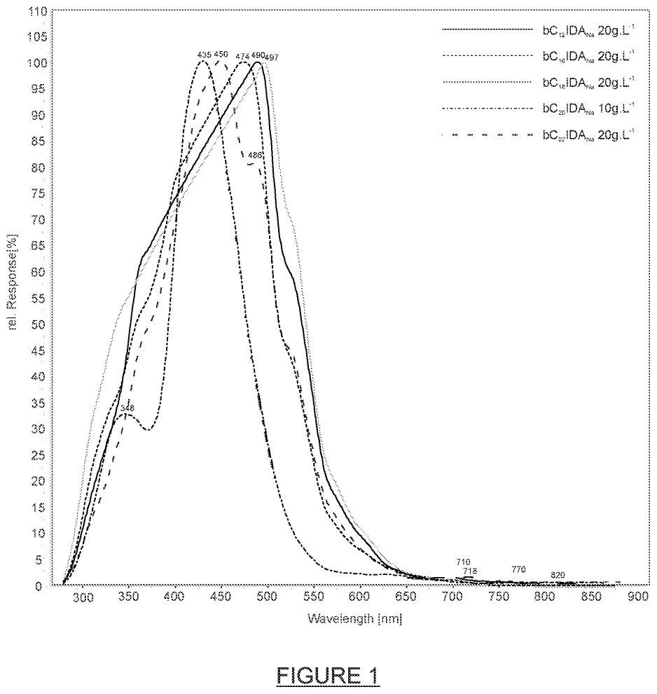

Non-limiting examples of preferred embodiments of the invention are described in more detail below, with reference to FIGS. 1 to 16.

The surfactant compounds in accordance with the present invention can be synthesized in terms of the following synthesis routes.
AC=aldol condensation
AH=acid hydrolysis Am=amidation
[dH]=dehydrogenation
E=esterification (M=R)
G=Guerbet reaction
HAM=hydroaminomethylation
HC=hydrocyanation
HF=hydroformylation
[H]=hydrogenation
MC=methoxycarbonylation
[O]=oxidation
OM=olefin (alkene) metathesis
(i)=reductive amination
(ii)=amidation via acid halide
D=O, NH₍₁₋ₓ₎Rₓ
M=metal, alkyl
R, R′, R″=alkyl, etc.
X=CH₂ (amine), C═O (amide)

The source of olefins (alkenes) can be industrial (i.e. Fischer-Tropsch synthesis, oligomerization of ethene, alkane (paraffin) cracking, hydrogenation of alkynes, etc.) or natural (i.e. oleochemicals, sugars, etc.).

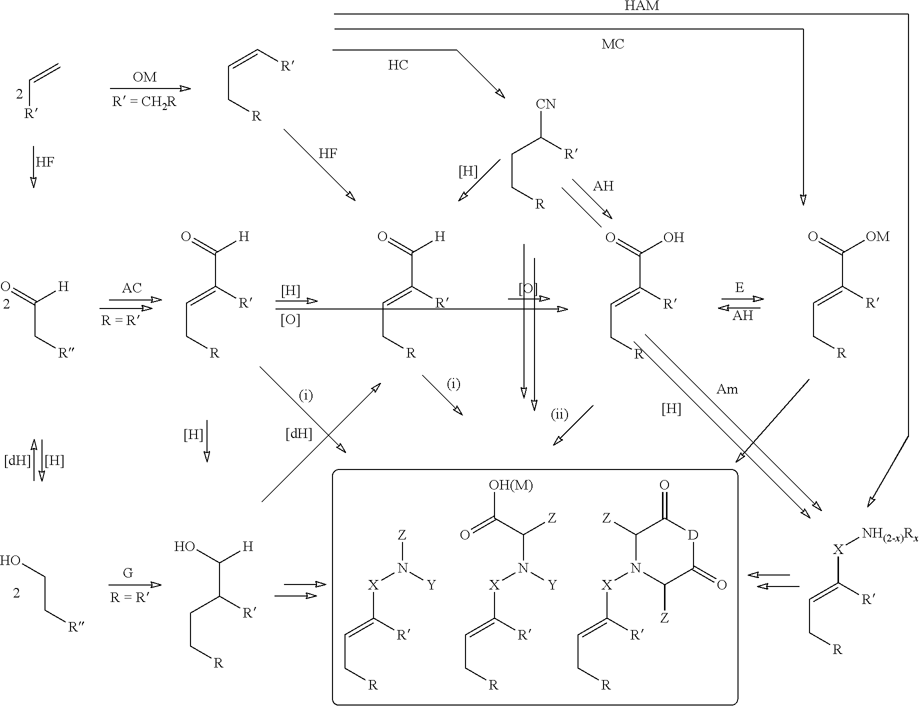

The surfactant compounds in accordance with the first aspect of the present invention, as synthesized from the above synthesis route, are shown below in Table 1.

TABLE 1
Surfactant compounds in accordance with the first aspect of the present invention

| | | | Synthesised and |
|---|---|---|---|
| | Y | Z | characterised |
| 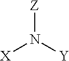 | H | H | |
| | H | R′ | |
| | H | (CH₂)ₙA | n = 0: bCₙp-ABA |
| | | | n = 2, A = OH,Cl: |
| | | | bCₙmEA, |
| | | | bCₙmEC |
| | R′ or X | (CH₂)ₙA | |
| | R′ or X | (CH₂)ₙCO₂M | |
| | R′ or X | CH₂CH(CH₃)O(CH₂CH₂O)ₙH | |
| | (CH₂)ₙA | (CH₂)ₙA | n = 2, A = OH,Cl: |
| | | | bCₙdEA, bCₙdEC |
| | (CH₂)ₙCO₂M | (CH₂)nCO₂M | |
| | CH₂CH(CH₃)O(CH₂CH₂O)ₙH | CH₂CH(CH₃)O(CH₂CH₂O)ₙH | |

X = COR or CH₂R
R = R′CH(CH₂)₂R′ or R′CCHCH₂R′
R′= alkyl (CₙH₂ₙ₊₁; n = 1, 2, ...)
n = 0, 1, 2, ...
A = OH, OR′, SH, SR′, NH₂, NHR′, NR′₂, CONH₂, CONHR′, CONR′₂, aromatic groups (e.g. C₆H₅, C₆H₄OH, C₆H₄COOH, etc.), heteroaromatic groups, halogens (F, Cl, Br, I)
M = H, R′, Li, Na, K, Rb, Cs, Er, Gd
bCₙ = R(CH₂)₂CHRCH₂—; bCₙ(O) = R(CH₂)₂CHRC(═O)—
beCₙ = R(CH₂)₂CHRCH₂—; beCₙ(O) = R(CH₂)₂CHRC(═O)—
p-ABA = para-aminobenzoic acid,
mEA = monoethylenealcohol,
dEA = diethylenealcohol,
mEC = monoethylenechloride,
dEC = diethylenechloride

The surfactant compounds in accordance with the second aspect of the present invention, as synthesized from the above synthesis route, are shown below in Table 2.

TABLE 2
Surfactant compounds in accordance with the second aspect of the present
invention

| | Y | Z | Synthesised and characterized |
|---|---|---|---|
| 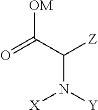 | H | H | bCₙGLY_{M} / bCₙ(O)GLY_{M}; ebCₙGLY_{M} / ebCₙ(O)GLY_{M} |
| | H | R′ | R′ = CH₃: bCₙALA_{M} / bCₙ(O)ALA_{M}; |
| | | | ebCₙALA_{M} / ebCₙ(O)ALA_{M} |
| | H | (CH₂)ₙA | n = 0: bCₙp-ABA n = 2, A = OH, Cl: bCₙmEA, |
| | | | bCₙmEC |
| | H | (CH₂)ₙCO₂M | n = 1: bCₙASP_{M} / bCₙ(O)ASP_{M}, ebCₙASP_{M} / |
| | | | ebCₙ(O)ASP_{M}; n = 2 : bCₙGLU_{M} / |
| | | | bCₙ(O)GLU_{M}; |
| | R′ or X | H | R′ = CH₃: bCₙSAR_{M} / bCₙ(O)SAR_{M}; |
| | | | ebCₙSAR_{M} / ebCₙ(O)SAR_{M} |
| | R′ or X | R′ | |
| | R′ or X | (CH₂)ₙA | |
| | R′ or X | (CH₂)ₙCO₂M | n = 1: bCₙASP_{M} / bCₙ(O)ASP_{M}, ebCₙASP_{M} / |
| | | | ebCₙ(O)ASP_{M} |
| | (CH₂)ₙA | H | |
| | (CH₂)ₙA | R′ | |
| | (CH₂)ₙA | (CH₂)ₙA | |
| | (CH₂)ₙA | (CH₂)ₙCO₂M | |
| | (CH₂)ₙCO₂M | H | n = 1: bCₙIDA_{M} / bCₙ(O)IDA_{M}; ebCₙIDA_{M} / |
| | | | ebCₙ(O)IDA_{M} |
| | (CH₂)ₙCO₂M | R′ | |
| | (CH₂)ₙCO₂M | (CH₂)ₙA | |
| | (CH₂)ₙCO₂M | (CH₂)ₙCO₂M | |

X = COR or CH₂R
R = R′CH(CH₂)₂R′ or R′CCHCH₂R′
R′ = alkyl (CₙH₂ₙ₊₁; n = 1, 2, ... )
n = 0, 1, 2, ...
A = OH, OR′, SH, SR′, NH₂, NHR′, NR′₂, CONH₂, CONHR′, CONR′₂, aromatic groups (e.g. C₆H₅, C₆H₄OH, C₆H₄COOH, etc.), heteroaromatic groups, halogens (F, Cl, Br, I)
M = H, R′, Li, Na, K, Rb, Cs, Er, Gd
bCₙ = R(CH₂)₂CHRCH₂—; bCₙ(O) = R(CH₂)₂CHRC(═O)—
beCₙ = R(CH₂)₂CHRCH₂—; beCₙ(O) = R(CH₂)₂CHRC(═O)—
ALA = alanine;
ASP = aspartic acid;
GLU = glutamic acid;
GLY = glycine;
IDA = iminodiacetic acid;
SAR = sarcosine.

The surfactant compounds in accordance with the third aspect of the present invention, as synthesized from the above synthesis route, are shown below in Table 3.

TABLE 3
Surfactant compounds in accordance with the third and fourth aspect of the present
invention

| | | Synthesised and |
|---|---|---|
| | Z | characterized |
| 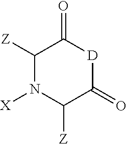 | H | D = O: bCₙMPD₂₆ |
| 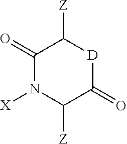 | R′(CH₂)ₙA | |
| | (CH₂)ₙCO₂M | |

X = COR or CH₂R
R = R′CH(CH₂)₂R′ or R′CCHCH₂R′
R′ = alkyl (CₙH₂ₙ₊₁; n = 1, 2, ... )
D = O, N(CH₂)ₙA, N(C₆H₄)ₙA
n = 0, 1, 2, ...
A = OH, OR′, SH, SR′, NH₂, NHR′, NR′₂, CONH₂, CONHR′, CONR′₂, aromatic groups (e.g. C₆H₅, C₆H₄OH, C₆H₄COOH, etc.), heteroaromatic groups, halogens (F, Cl, Br, I)M = H, R′, Li, Na, K, Rb, Cs, Er, Gd
MPD₂₆ = morpholine-2,6-dione

The surfactant compounds in accordance with the first, second, third and fourth aspects of the present invention demonstrated the following surfactant properties as shown in Table 4.

TABLE 4
Surfactant Properties

| | Surfactant properties | | | | |
|---|---|---|---|---|---|
| | CMC | [C₂₀] | Π_{CMC} | CMC | γ_{CMC} |
| Surfactant | (mmol/L) | (mmol/L) | (mN/m) | (mg/L) | (mN/m) |
| bC₁₂IDA_{Na} | 99.014 | 2.089 | 40.823 | 34357.84 | 30.71 |
| bC₁₆IDA_{Na} | 3.846 | 0.192 | 42.918 | 1542.20 | 28.17 |
| bC₁₈IDA_{Na} | 0.711 | 0.016 | 45.400 | 305.03 | 27.37 |
| bC₂₀IDA_{Na} | 0.142 | 0.005 | 42.715 | 64.83 | 28.89 |
| bC₁₂ASP_{Na} | 3.412 | 0.169 | 47.128 | 1177.18 | 26.27 |
| bC₁₆ASP_{Na} | 5.132 | 0.043 | 46.031 | 2068.13 | 25.73 |
| bC₁₈ASP_{Na} | 0.826 | 0.013 | 46.309 | 354.17 | 25.33 |
| bC₂₀ASP_{Na} | 0.017 | 0.001 | 46.727 | 7.86 | 25.20 |
| bC₁₈(O)ASP_{Na} | 10.776 | 0.561 | 40.354 | 4776.51 | 31.21 |
| bC₁₈(O)GLU_{Na} | 10.987 | 0.469 | 37.875 | 5020.94 | 33.64 |
| bC₁₈(O)ALA_{Na} | 1.310 | 0.092 | 43.852 | 493.77 | 30.26 |
| bC₁₈(O)GLY_{Na} | 1.005 | 0.095 | 45.360 | 364.77 | 28.39 |
| bC₁₂(O)ALA_{Na} | 43.974 | 0.405 | 42.473 | 12884.36 | 27.21 |
| bC₁₂(O)GLY_{Na} | 54.575 | 0.095 | 39.553 | 15226.32 | 31.55 |
| bC₁₂(O)GLU_{Na} | 41.317 | 4.941 | 39.107 | 15411.31 | 30.22 |
| bC₁₂(O)IDA_{Na} | 30.115 | 0.741 | 39.400 | 11232.84 | 30.06 |
| bC₁₆(O)IDA_{Na} | 30.697 | 0.106 | 41.853 | 13168.92 | 29.66 |
| bC₁₆(O)ALA_{Na} | 5.967 | 0.004 | 43.452 | 2082.42 | 28.06 |
| bC₁₆(O)ASP_{Na} | 10.796 | 0.344 | 42.807 | 4480.24 | 28.44 |
| bC₁₆(O)GLU_{Na} | 24.787 | 0.332 | 41.594 | 10633.55 | 29.71 |
| bC₁₆(O)GLY_{Na} | 4.544 | 0.180 | 42.828 | 1522.09 | 28.68 |

CMC = Critical micelle concentration
[C₂₀] = Surfactant concentration when surface tension has reduced by 20 mN/m (at ca. 52 mN/m)
Π_{CMC} = Surface excess at CMC
γ_{CMC} = Surface tension at CMC

From the above results, it can be seen that the surfactant compounds according to the first and second aspects of the invention have been synthesised which demonstrate superior surfactant properties in comparison to known surfactants.

Non-limiting examples of the compounds in accordance with the second aspect of the invention illustrating the IUPAC names and abbreviations are presented herein below.

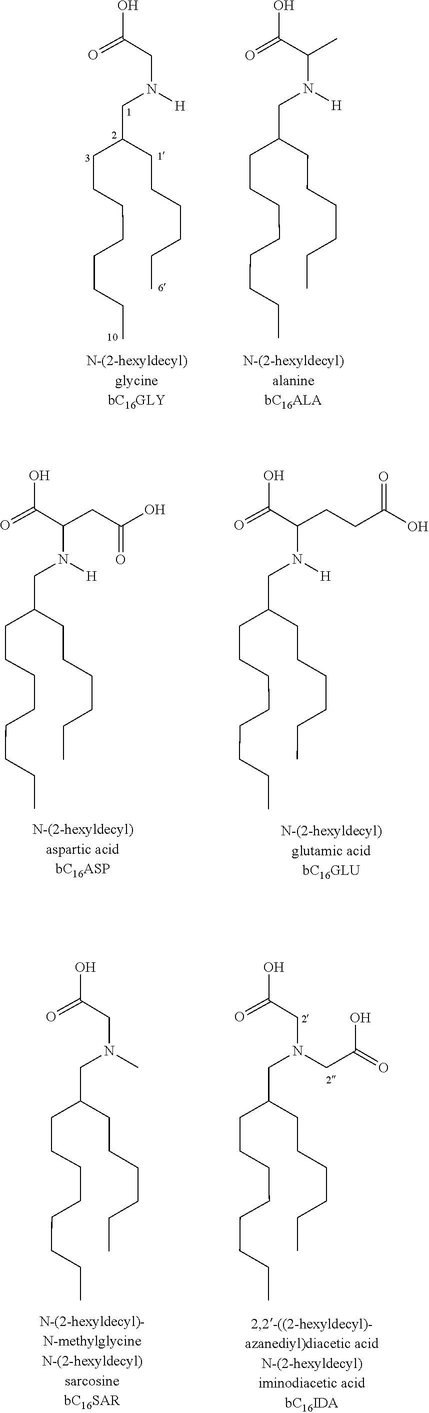 [SMILES: CCCCCCCCC(CCCCCC)CNCC(=O)O ; CCCCCCCCC(CCCCCC)CNC(C)C(=O)O ; CCCCCCCCC(CCCCCC)CNC(CC(=O)O)C(=O)O ; CCCCCCCCC(CCCCCC)CNC(CCC(=O)O)C(=O)O ; CCCCCCCCC(CCCCCC)CN(C)CC(=O)O ; CCCCCCCCC(CCCCCC)CN(CC(=O)O)CC(=O)O]

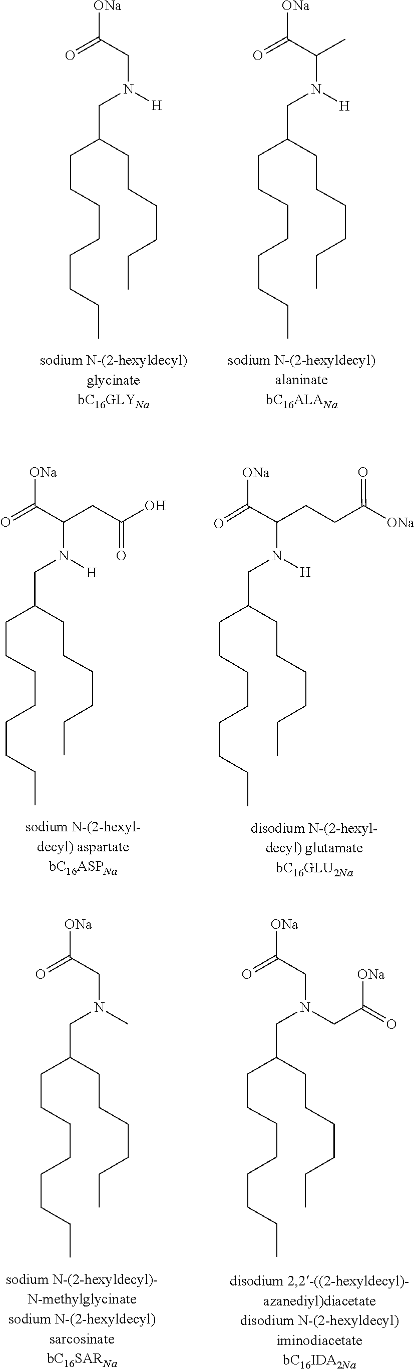

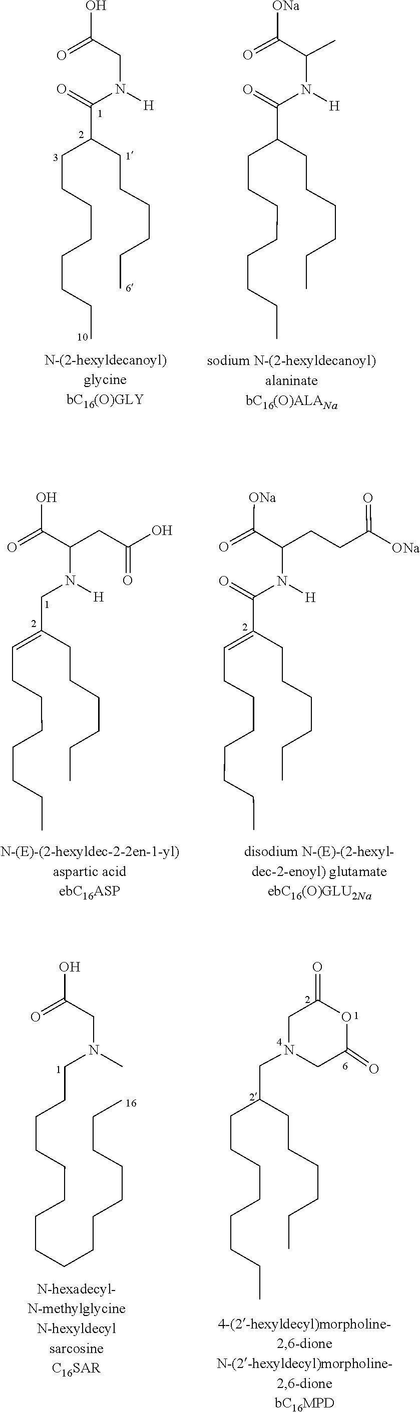

The surfactant compounds in accordance with the first aspect of the present invention, as synthesized from the above synthesis route, have now been found to luminesce as shown under in Table 5.

TABLE 5
Luminescent surfactant compounds in accordance with the first aspect of the
present invention

| | | | Synthesised and |
|---|---|---|---|
| | | | show |
| | Y | Z | luminescence |
| 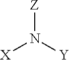 | H | H / R′ / (CH₂)ₙA | n = 0: bCₙp-ABA n = 2, A = OH,Cl: bCₙmEA, bCₙmEC |
| | R′ / X / (CH₂)ₙA | (CH₂)ₙA | n = 2, A = OH,Cl: |
| | | | bCₙdEA, bCₙdEC |
| | | (CH₂)ₙCO₂M | |
| | | CH₂CH(CH₃)O(CH₂CH₂O)ₙH | |
| | (CH₂)ₙCO₂M | (CH₂)ₙCO₂M | |
| | CH₂CH(CH₃)O(CH₂CH₂O)ₙH | CH₂CH(CH₃)O(CH₂CH₂O)ₙH | |

X = COR or CH₂R
R = R′CH(CH₂)₂R′; or R′CCHCH₂R′
R′ = alkyl (CₙH₂ₙ₊₁; n = 1, 2, ...)
n = 0, 1, 2, ...
A = OH, OR′, SH, SR′, NH₂, NHR′, NR′₂, CONH₂, CONHR′, CONR′₂, aromatic groups (e.g. C₆H₅, C₆H₄OH, C₆H₄COOH, etc.), heteroaromatic groups, halogens (F, Cl, Br, I)
M = H, R′, Li, Na, K, Rb, Cs, Er, Gd

The surfactant compounds in accordance with the second aspect of the present invention, as synthesized from the above synthesis route, have now been found to luminesce as shown below in Table 6.

TABLE 6
Luminescent surfactant compounds in accordance with the second aspect of the
present invention

| | Y | Z | Synthesised and show luminescence |
|---|---|---|---|
| 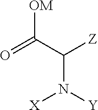 | | | bCₙGLY_{M} / bCₙ(O)GLY_{M}; ebCₙGLY_{M} / ebCₙ(O)GLY_{M} |
| | H | H / R′/ | R′ = CH₃: bCₙALA_{M} / bCₙ(O)ALA_{M}; |
| | | | ebCₙALA_{M} / ebCₙ(O)ALA_{M} |
| | | (CH₂)ₙA / | n = 0: bCₙp-ABA n = 2, A = OH, Cl: bCₙmEA, |
| | | (CH₂)ₙCO₂M | bCₙmEC |
| | | | n = 1: bCₙASP_{M} / bCₙ(O)ASP_{M}, ebCₙASP_{M} / |
| | | | ebCₙ(O)ASP_{M}; n = 2: bCₙGLU_{M} / |
| | | | bCₙ(O)GLU_{M}; |
| | | H | R′ = CH₃: bCₙSAR_{M} / bCₙ(O)SAR_{M}; |
| | | | ebCₙSAR_{M} / ebCₙ(O)SAR_{M} |
| | R′/X / | R′ | |
| | (CH₂)ₙA | (CH₂)ₙA | |
| | | (CH₂)ₙCO₂M | n = 1: bCₙASP_{M} / bCₙ(O)ASP_{M}, ebCₙASP_{M} / |
| | | | ebCₙ(O)ASP_{M} |
| | (CH₂)ₙCO₂M | H / R′/ | n = 1: bCₙIDA_{M} / bCₙ(O)IDA_{M}; ebCₙIDA_{M} / |
| | | (CH₂)ₙA / | ebCₙ(O)IDA_{M} |
| | | (CH₂)ₙCO₂M | |

X = COR or CH₂R
R = R′CH(CH₂)₂R′ or R′CCHCH₂R′
R′ = alkyl (CₙH₂ₙ₊₁; n = 1, 2, ...)
n = 0, 1, 2,
A = OH, OR′, SH, SR′, NH₂, NHR′, NR′₂, CONH₂, CONHR′, CONR′₂, aromatic groups (e.g. C₆H₅, C₆H₄OH, C₆H₄COOH, etc.), heteroaromatic groups, halogens (F, Cl, Br, I)
M = H, R′, Li, Na, K, Rb, Cs, Er, Gd

The surfactant compounds in accordance with the third aspect of the present invention, as synthesized from the above synthesis route, have now been found to luminesce as shown below in Table 7.

TABLE 7
Luminescent surfactant compounds in accordance with the third and fourth aspect
of the present invention

| | | Synthesised and show |
|---|---|---|
| | Z | luminescence |
| 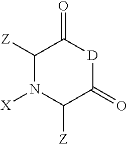 | H | D = O: bCₙMPD₂₆ |
| 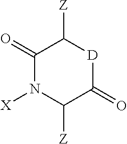 | R′ | |
| | (CH₂)ₙA | |
| | (CH₂)ₙCO₂M | |

X = COR or CH₂R
R = R′CH(CH₂)₂R′ or R′CCHCH₂R′
R′ = alkyl (CₙH₂ₙ₊₁; n = 1, 2, ...)
D = O, N(CH₂)ₙA, N(C₆H₄)ₙA
n = 0, 1, 2, ...
A = OH, OR′, SH, SR′, NH₂, NHR′, NR′₂, CONH₂, CONHR′, CONR′₂, aromatic groups (e.g. C₆H₅,
C₆H₄OH, C₆H₄COOH, etc.), heteroaromatic groups, halogens (F, Cl, Br, I)
M = H, R′, Li, Na, K, Rb, Cs, Er, Gd

The luminescent surfactant compounds in accordance with the first, second, third and fourth aspects of the present invention demonstrated the following spectral properties as shown in FIGS. 1-16 and in Table 8.

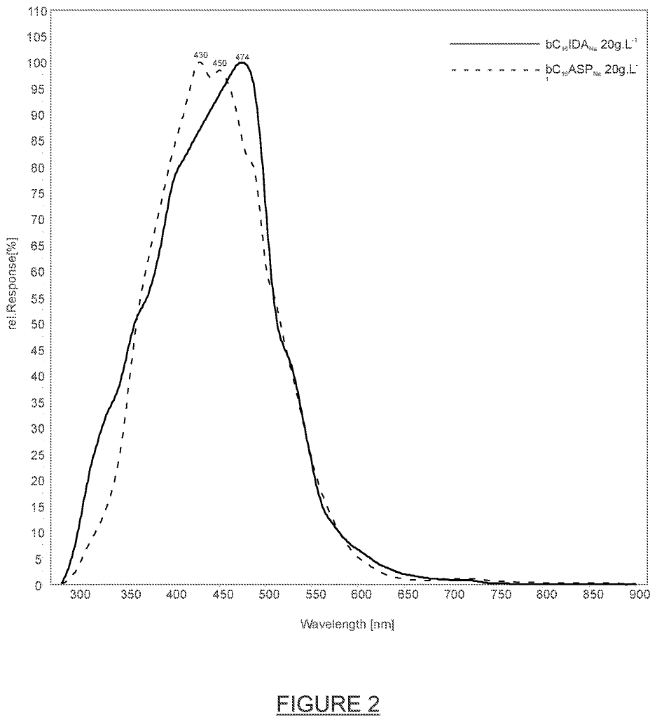

The typical emission wavelengths of the surfactants of the present invention are shown in FIG. 1 and FIG. 2. Surfactants according to the first and second aspects of the invention exhibit luminescence under certain conditions. It was also observed that certain surfactants luminesce more than others. In particular, it was found that surfactants containing dense electron rich groups luminesce more. Therefore, surfactants such as the IDA- and ASP-amine surfactants luminesce more than the amine or amide-alcohol group surfactants. The typical emission wavelengths of the surfactants of the present invention are as follows:

| | bC₁₆ASP | bC₁₂IDA | bC₁₆IDA | bC₁₈IDA | bC₂₀IDA | bC₂₂IDA |
|---|---|---|---|---|---|---|
| Emission λₘₐₓ/nm | 430 and 450 | 490 | 474 | 497 | 435 | 450 |

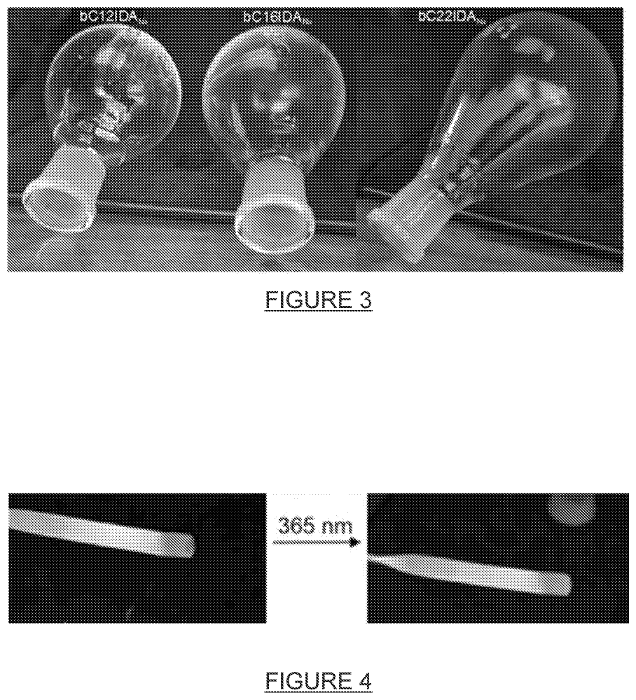

Surfactants with the same head group and with different tail lengths were found to exhibit a slight change in the emitted wavelength. A change in tail length implies a variation in the amount of methylene groups in the hydrocarbon chain of the tail, as shown in FIG. 3.

Surfactants isolated as HCl salts or as waxes of the zwitterionic compounds also fluoresces in the solid state. Furthermore, the wavelengths of the absorbed UV and the emitted light differs with the concentration and changes to the structure of the surfactants. The surfactants are isolated via an acid extraction method. Depending on the chain length of the tail group, a wax is obtained, as shown in FIG. 4.

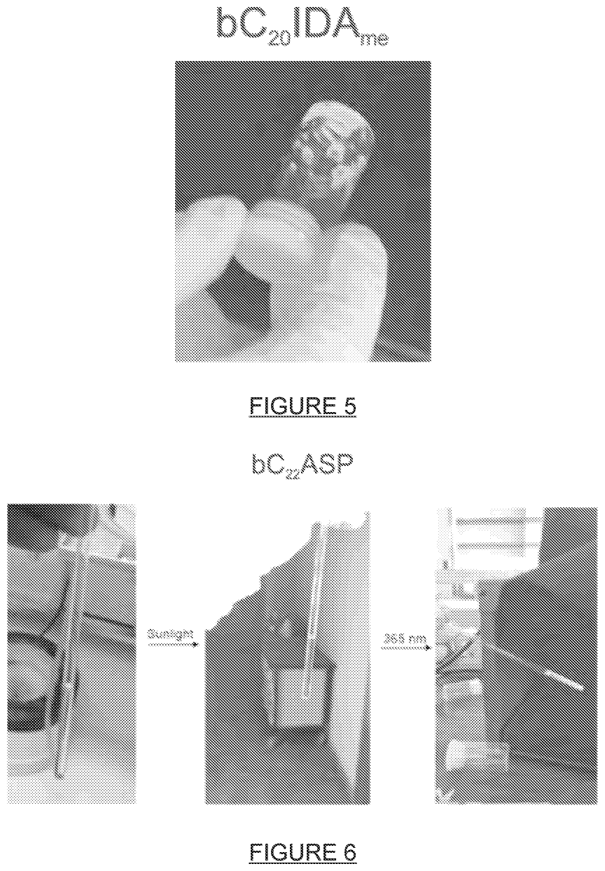

The wavelength of the emitted light changes more prominently when the head group structure changes. Methyl esters emit a lower energy light, i.e., a green light, as demonstrated in FIG. 5.

When the surfactants are dissolved in an organic solvent, the luminescence changes. Therefore, the wavelength of emission is not only influenced by the fluorophore but by the solvent and concentration of the surfactant. As shown in FIG. 6, the wavelength of the emitted light also depends on the source of the radiation.

The intensity of the luminescence also varies with different organic solvents. It was found that the following solvents enhanced the luminescence: DMSO, MeOH and CHCl₃ (other solvents may also enhance the luminescence).

The intensity of the fluorescence is quenched when the surfactants are isolated as Na-salts and when the surfactants are dissolved in water.

TABLE 8
Spectral Properties

| | Surfactant properties | | Luminescent data | | | | | |
|---|---|---|---|---|---|---|---|---|
| | CMC | γ_{CMC} | | | | | | Conc. |
| Surfactant | {mmol/L} | (mN/m) | λ_{ab} | λₑₘ | AUₘ | ∫ₑₘ | Solvent | (mmol/L) |
| bC₁₂ABA (s) | | | | 463.93 | 7987.92 | 7987.92 | | |
| bC₁₈GLY (s) | | | | 416.00 | 320.38 | 320.38 | | |
| bC₁₆IDA (s) | | | | 491.00 | 307.91 | 307.91 | | |
| bC₂₂IDA (s) | | | | 492.00 | 254.46 | 254.46 | | |
| bC₁₂IDA_{Na} | 99.014 | 30.71 | 350 | 433 | 11.929 | 1462.9 | H₂O | 100 |
| bC₁₂IDA | | | 340 | 414 | 28.273 | 3737.1 | DMSO | 500 |
| bC₁₂IDA | | | 280 | 418 | 54.496 | 7029.3 | MeOH | 1000 |
| bC₁₂p-ABA | | | 320 | 420 | 724.376 | 48491.1 | MeOH | 6.125 |
| bC₁₆MPD₂₅ | | | 410 | 483 | 25.456 | 2515.1 | MeOH | 125 |

Table 8 illustrates several key parameters: λ_{ab}, the wavelength at which maximum absorption occurs; λₑₘ, the wavelength of maximum emission; AUₘ, representing arbitrary units of maximum emission; and∫ₑₘ, indicating the integrated area of emission.

As shown in Table 8, λ_{ab} provides insight into where light is absorbed and can therefore be used to characterise the efficiency of a material to be used as light filters at specific wavelengths. λₑₘ represents the wavelength at which the maximum emission is detected, a crucial factor in various applications, including UV-downconverters. Considering that solar panels exhibit an optimal wavelength efficiency for the conversion of light into electricity, understanding λₑₘ is essential for optimising their solar cell performance. AUₘ provides a measure of the light intensity at a given wavelength and∫ₑₘ offers insights into total emitted photons, both metrics aiding in the assessment of emission efficiency.

UV-visible and fluorescence analyses were conducted on several surfactants, revealing that bC₁₂p-ABA displayed the highest emission efficiency. Nonetheless, aliphatic surfactants also exhibit fluorescence in both solid-state and various solvents. The emission intensity (AUₘ) and integrated emission (∫ₑₘ) vary depending on the solvent and the surfactant's state. In terms of the data provided here, it has been observed that the tail group does not necessarily affect the wavelength of maximum emission (λₑₘ). However, the head group, pH, and chelated ions do influence λₑₘ.

These results exemplified in Table 8 indicate that the presence of a particular tail group within a surfactant does not invariably impact λₑₘ. However, the head group's composition, the pH, and the presence of chelated ions appear to modify the wavelength of maximum emission.

Based on the results above, the Applicant believes that the luminescent surfactant compounds according to the first, second, third and fourth aspects described in the present invention are capable of being incorporated in the development of pH sensors or detectors for specific ions such as potassium (K⁺), sodium (Na⁺), calcium (Ca²⁺), magnesium (Mg²⁺), copper (Cu²⁺), erbium (Er³⁺), and gadolinium (Gd³⁺).

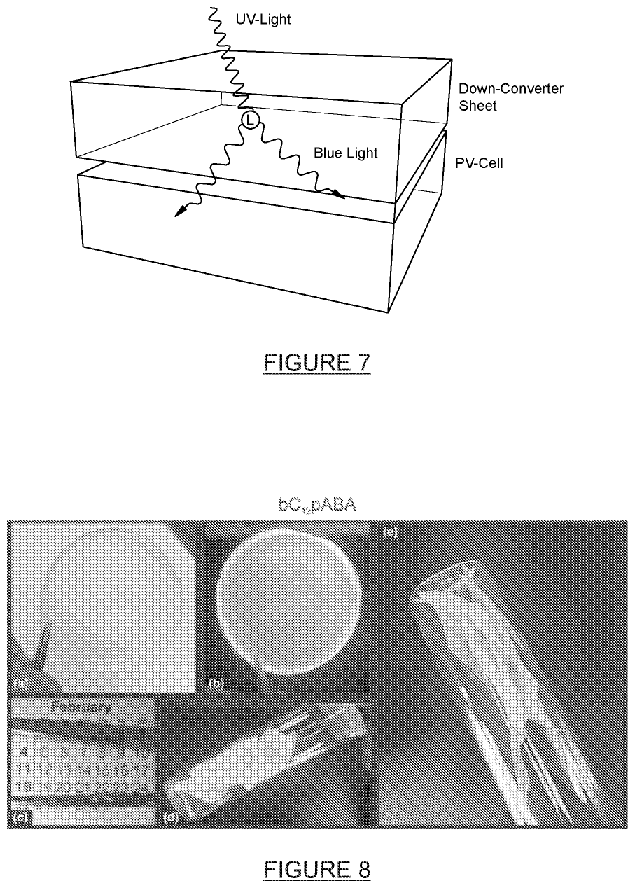

FIG. 7 demonstrates the concept of using luminescent surfactant compounds according to the first, second, third and fourth aspects described in the present invention instead of quantum dots to down convert UV-light to blue light.

The Applicant believes that this will increase the amount of light that is within the absorbable range of the photovoltaic cell (PV-cell), thereby increasing the power conversion efficiency (PCE) of a PV-cell.

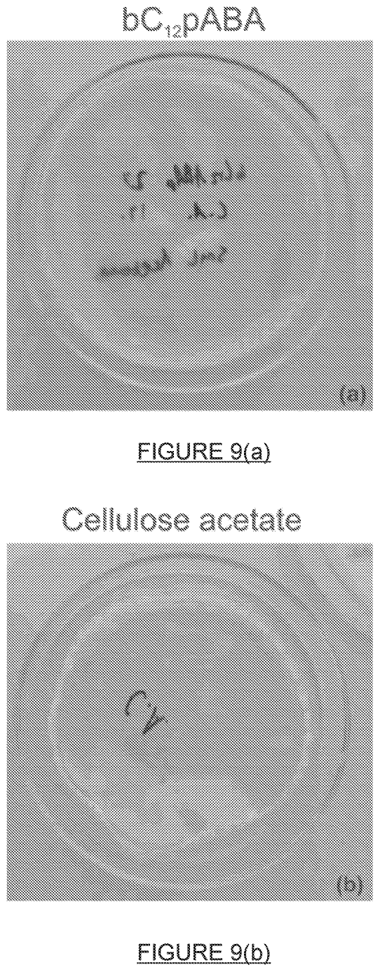

FIG. 8 illustrates a cellulose acetate (CA) sheet impregnated with bC₁₂p-ABA. It is also noted that all the aliphatic amino acid surfactants exhibit fluorescence both as solids and in various solvents. Importantly, the emission intensity, as quantified by AUₘ, and the integrated emission, as represented by ∫ₑₘ, are influenced by the solvent medium and the physical state of the surfactant.

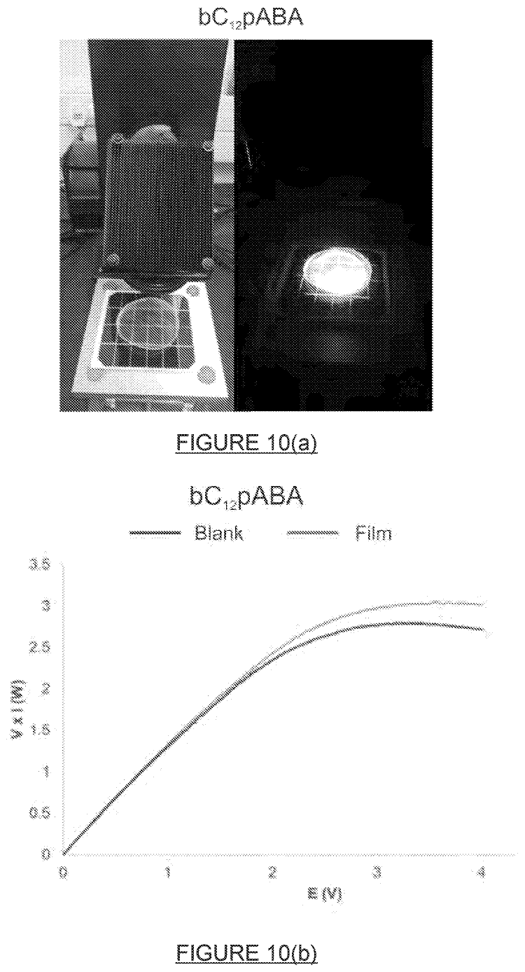

As shown in FIG. 9 and FIG. 10, the bC₁₂p-ABA improves the morphological properties of the polymer film, improving its spreadability, elasticity and thereby reducing the contraction of the polymer during solvent evaporation. Polymer coatings with a homogenous surface texture are necessary for reducing the reflectance of luminescent solar concentrators (LSCs).

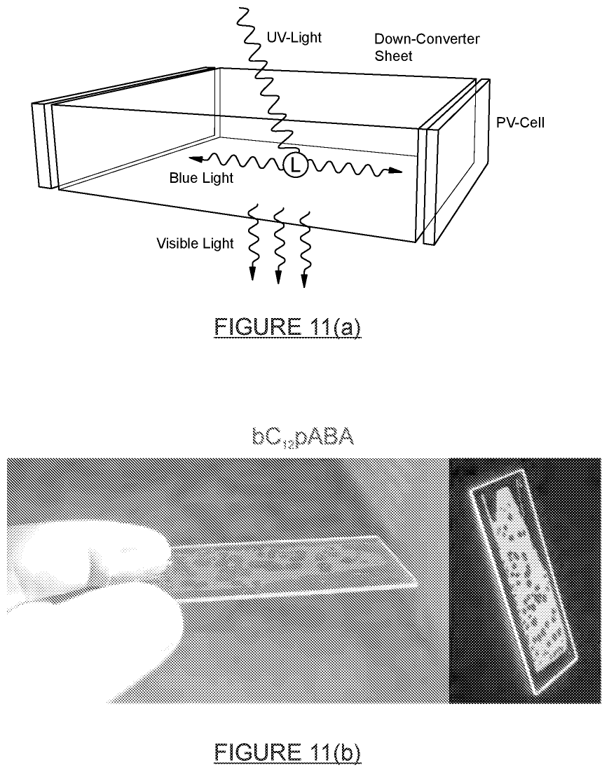

FIG. 11 illustrates how UV-light is down converted into blue light and travels via a wave guide to the solar cells on the edges of the LSC. This is of economic importance, as it implies that a greater surface area is available for light capture while a smaller solar cell can be used to capture the light. As the LSC is based on luminescent surfactant compounds according to the first, second, third and fourth aspects of the present invention and not quantum dots the transparency of the film for visible light is higher and therefore these LSCs may find application in agrivoltaics.

The surfactant compounds in accordance with the fifth aspect of the present invention have now been found to luminesce as shown below in Table 9.

TABLE 9
Luminescent surfactant compounds in accordance with the fifth aspect of the
present invention

| | | | Synthesised | |
|---|---|---|---|---|
| | | | and show | Comercially |
| | Y | Z | luminescence | available |
| 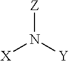 | H | H / R′ / (CH₂)ₙA | | |
| | R′ | R′ / (CH₂)ₙA | | |
| | (CH₂)ₙA | (CH₂)ₙA | | |
| | (CH₂)ₙCO₂M | (CH₂)ₙCO₂M | | |
| | CH₂CH(CH₃)O(CH₂CH₂O)ₙH | CH₂CH(CH₃)O(CH₂CH₂O)ₙH | | |
| 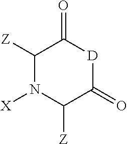 | H | H / R′ / (CH₂)ₙA / (CH₂)ₙCO₂M | | Galsoft, Yeser |
| | R′ / (CH₂)ₙA | H / R′ / (CH₂)ₙA / | | Crodasinic, |
| | | (CH₂)ₙCO₂M | | Yeser, Galsoft |
| | (CH₂)ₙCO₂M | H / R′ / (CH₂)ₙA / | n = 1: CₙIDA_{M} | Amphokem ™ |
| | | (CH₂)ₙCO₂M | | CADP |
| 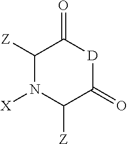 | 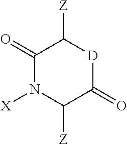 | H | | |
| | | R′ | | |
| | | (CH₂)ₙA | | |
| | | (CH₂)ₙCO₂M | | |

X = COR or CH₂R
R = R′ or CHCHR′
R′ = alkyl (CₙH₂ₙ₊₁; n = 1, 2, ...)
D = O, N(CH₂)ₙA, N(C₆H₄)ₙA
n = 0, 1, 2,
A = OH, OR′, SH, SR′, NH₂, NHR′, NR′₂, CONH₂, CONHR′, CONR′₂, aromatic groups (e.g. C₆H₅, C₆H₄OH, C₆H₄COOH, etc.), heteroaromatic groups, halogens (F, Cl, Br, I)
M = H, R′, Li, Na, K, Rb, Cs, Er, Gd

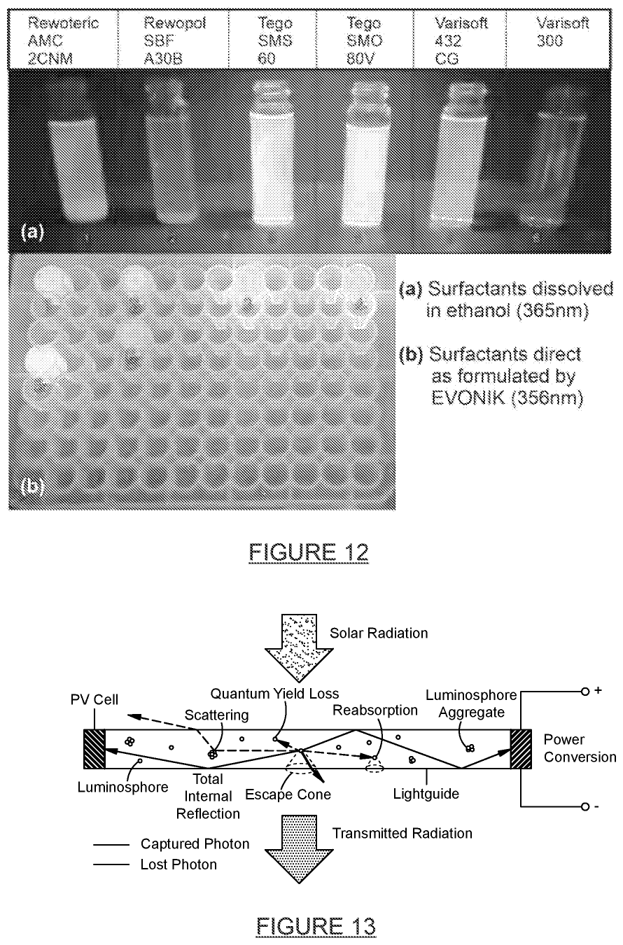

As shown in FIG. 12, linear heteroatom functionalized surfactant compounds according to the fifth aspect of the present invention have for the first time in the art been shown to luminesce.

Secondary properties arise when aggregation occurs, for example, non-traditional intrinsic luminescence (NTIL) of aggregated surfactants. Four classes of NTIL have been identified. The first group consists of silicone and siloxane based NTIL. The second group consists of dendrimer and polymer based NTIL. The third group consists of biopolymer, protein and conjugated polymer based NTIL. The fourth group consists of cross linked, non-conjugated polymer dot based NTIL. Table 9 below summarises non-traditional luminescent materials (NTLMs) and their principal photophysical advantages relative to many dyes and Fps.

TABLE 9
Non-Traditional Luminescent Materials (NTLMs)

| | | |
|---|---|---|
| NTLM (and size) | Common material(s) | Potential advantages |

Luminescent Nanoparticles

| | | |
|---|---|---|
| Quantum dots (1-10 nm) | CdX, PbX, InP, CIS, AgX and Si | Better brightness via downshifting or |
| | nanocrystals; some materials | 2PE; little or no photobleaching; |
| | include thin shell(s) of other | simultaneous excitation of multiple |
| | semiconductor material(s) | colours; narrow emission spectra; some |
| | | materials have NIR-I and NIR-II |
| | | emission |
| Conjugated polymer | Aggregates of conjugated | Superior brightness; more resistant to |
| nanoparticles CPNs and | polymers (and often amphiphilic | photobleaching; can be nonblinking |
| polymer dots Pdots (4-100 | polymers), such as those based | |
| nm) | on thiophene, phenylenevinylene, | |
| | and phenyleneethylene | |
| | monomers | |
| Single-walled carbon | Chiral rolled sheets of | Nonphotobleaching; nonblinking; |
| nanotubes SWCNTs | graphene, <2 nm in diameter, | narrow excitation and emission bands; |
| (10⁻²-10⁻³ nm) | variable length | NIR-I and NIR-II emission |
| Carbon dots (1-10 nm) | Nanoparticles with amorphous | Better brightness via downshifting or |
| | and graphitic carbon, oxidised | 2PE; more resistant to photobleaching |
| | surface regions | |
| Graphene dots (<20 nm) | Fragment of the graphene sheet | Better brightness via downshifting or |
| | with oxidised edges | 2PE; more resistant to photobleaching |
| Nanodiamonds (4-10² nm) | Nanoparticles with diamond-like | Nonphotobleaching; nonblinking |
| | lattice and luminescent vacancies; | |
| | oxidised surface | |
| Metal nanoclusters (<3 nm) | Clusters of tens to hundreds of | Better brightness by 2PE; more |
| | Au, Ag, and Cu atoms | resistant to photobleaching; some |
| | | materials have NIR emission |
| Upconversion nanoparticles | Rare earth fluorides (e.g., NaYF₄) | Upconversion luminescence (UCNPs) |
| UCNPs and downshifting | doped with trivalent lanthanide | or NIR-Il emission (DLNPs); narrow |
| lanthanide-based | ions (e.g., Yb³⁺, Er³⁺, Tm³⁺, Tb³⁺, | emission lines; long excited-state |
| nanoparticles DLNPs | Eu³⁺, Dy³⁺, Sm³⁺, Ho³⁺, Pr³⁺) | lifetimes; nonphotobleaching; |
| (10⁻¹-10² nm) | | nonblinking |
| Triplet-triplet annihilation | Rare earth metals doped with | Upconversion luminescence |
| upconversion TTA-NPs | photosensitisers (e.g., | |
| (10⁻¹-10² nm) | metalloporphyrins, halogenated | |
| | BODIPY dyes) and annihilators | |
| | (e.g., 9,10-diphenylanthracene, | |
| | rubrene, BODIPY dyes) | |
| Persistent luminescence | Metal oxides (e.g., ZnGa₂O₄) | Luminescence may persist up to hours |
| nanoparticles, pLNPs | doped with transition metal (e.g., | after photoexcitation; potential for |
| (5-10² nm) | Cr³⁺) or lanthanide (e.g., Eu^{2+/3+}) | upconversion luminescence; potential |
| | ions | for excitation of luminescence by X-ray |
| | | irradiation |

Molecular Materials

| | | |
|---|---|---|
| Luminescent lanthanide | Trivalent lanthanide ions (e.g., | Long excited-state lifetime (time- |
| complexes LLCs (<2 nm) | Tb³⁺, Eu³⁺, etc.) complexed by | gating); narrow emission lines; more |
| | organic ligands | resistant to photobleaching |
| Transition metal complexes | Transition metal ions (e.g., Ru²⁺, | Long excited-state lifetime |
| TMCs (<2 nm) | Ir³⁺, etc.) complexed by organic | (time-gating) |
| | ligands | |

### Luminescent Solar Concentrator (LSC) Media

In a luminescent solar concentrator (LSC) medium, light is trapped via total internal reflection and concentrated towards a PV cell located at the edges of the medium. However, the efficiency of LSCs is hindered by for example quantum yield losses, limited absorption by the luminophore, reabsorption of the photoluminescence by the luminophore, by scattering and the like. Incoming light will either be reflected at the surface of the concentrator medium or absorbed by a luminophore or transmitted through the LSC. Surfactants serving as luminophores not only increases the number of absorbed photons but also reduces the reflection on the LSCs by reducing surface inhomogeneities. When a luminophore absorbs a photon, it may either emit light via photoluminescence or dissipate energy in the form of heat. The likelihood of each outcome is determined by the photoluminescence quantum yield (PLQY) of the luminophore. The light emitted can undergo total internal reflection, be reabsorbed by another luminophore, or escape from the material, termed as “escape cone” loss, all of which depend on the emission angle of the light, as demonstrated in FIG. 13).

When a photon is reabsorbed, there is an added chance of experiencing photoluminescence quantum yield (PLQY) loss or “escape cone” loss. LSCs with considerable reabsorption tend to have significantly lower efficiencies as seen for some of the quantum dots. The impact of luminophore aggregation varies with the type of luminophore used. For instance, the aggregation of fluorescent dyes can lead to fluorescence quenching, which lowers the photoluminescence quantum yield (PLQY), or it may cause an increased Stokes shift, potentially diminishing reabsorption losses. On the other hand, poorly dispersed nanoparticles may cluster together, scattering light and decreasing the light transmitted to the device's edge.

The Applicant believes that luminescent surfactant compounds according to the present invention may not only decrease the amount of reflection (by preventing the clustering of nanoparticles) but may also synergistically increase the amount of absorbed and emitted photons in a LSC.

### Smart Materials

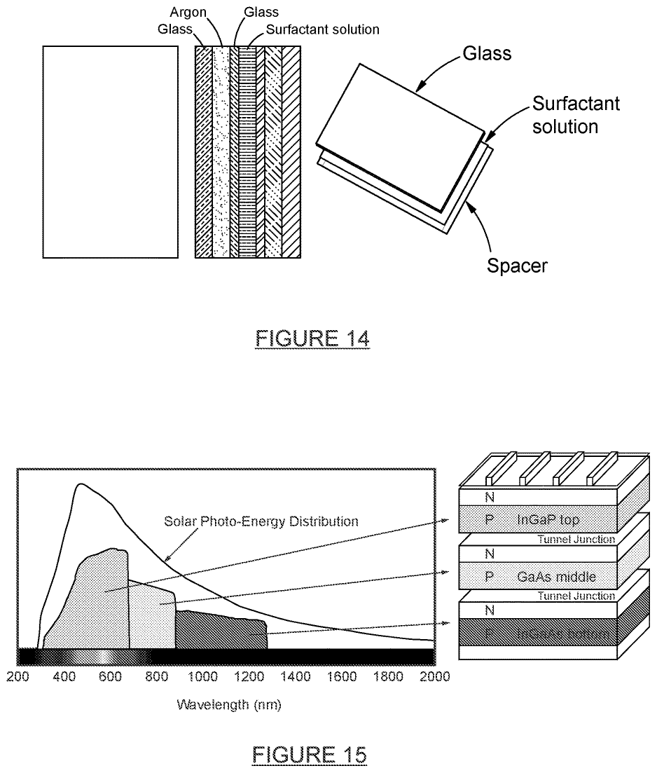

Surfactants are commonly used in smart windows as solar modulators to produce thermo responsive smart windows, as shown in FIG. 14. It is believed that these surfactants, used in smart windows, could lower the temperature in a building by a few degrees without any energy input. A drop in internal temperature of 1 °C would have a benefit on cost and electricity usage. However, the use of the AASs exhibiting NTIL could decrease the amount of UV radiation allowed into the building. Upon sun irradiation, the synthesised liquid crystal smart window will be excited into a coloured phase decreasing the amount of UV penetration. After the irradiation stops, the coloured window would return to colourless.

The Applicant is of the belief that the luminescent surfactant compounds of the present invention may be used as polymer coatings or sheets to be utilised to down-convert and guide high-energy light towards the edges of structures, such as a tunnel enclosure, for agricultural purposes. The concentrated light at the tunnel's edges could be collected using photovoltaic cells, where it is converted into electricity. Furthermore, transparent waveguide solar concentrators allow visible light to penetrate, promoting photosynthesis within the tunnel.

In addition to the agrivoltaics applications, incorporation of the luminescent surfactant compounds of the present invention into polyethylene sheets that are used in vegetable tunnels has interesting applications for crop production.

Here, the luminescent surfactant compounds of the present invention would be capable of absorbing UV light and re-emitting it as visible light, specifically blue light in this case. Blue light is a critical component of the light spectrum for plant growth, influencing various physiological processes including phototropism (directional growth in response to light), stomatal opening (which affects water loss and gas exchange), and photosynthesis.

This would provide beneficial application in achieving:

Enhanced Photosynthesis: Blue light is highly effective in driving photosynthesis; incorporating the luminescent surfactant compounds of the present invention could potentially increase the efficiency of photosynthesis, especially in environments where UV light is abundant but direct sunlight is partially filtered by the plastic.

Improved Plant Morphology: Blue light influences plant morphology, including reduced internodal spacing (the distance between branches), leading to more compact and sturdy plants. This can be particularly beneficial for certain crops that benefit from such growth patterns.

Optimized Light Spectrum: By converting UV light, which is less useful for most plants, into blue light, the overall light spectrum inside the polytunnel could be optimized for plant growth. This could lead to more efficient use of the available light energy and ultimately increase yields and influence time of harvest.

### UV-Downconverters

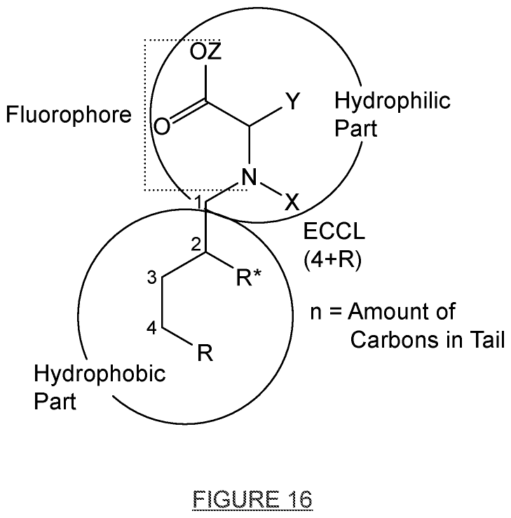

The principle of UV-downconverters is to lower the energy of radiation. UV-light from the sun decomposes certain materials. The efficiency of conversion of light to electricity via photo voltaic cells in solar panels is usually optimal between 400 and 600 nm. The high energy light<300 nm decomposes materials and is not converted to electricity. SHARP developed a solar panel with a conversion efficiency of 37.7% as shown in FIG. 15.

It is believed that the luminescent surfactant compounds of the present invention will play a crucial role in downconverter technology, especially when these luminescent surfactant compounds of the present invention would themselves act as luminophores.

Downconverter technology is often used in applications like photovoltaics or lighting, where it is crucial to convert high-energy photons (such as ultraviolet light) into lower-energy photons (such as visible light). When luminescent surfactant compounds of the present invention as luminophores are used in downconverter technology, they can enable or enhance the process of energy transfer and photon emission in several ways. Surfactants can organise themselves into various structures such as micelles or liquid crystals and they can adsorb onto curtain surfaces. Adsorbed surfactants can be functionalised to create hydrophobic and for example dust repellent surface coatings. In some systems, the aggregated surfactant structures can facilitate energy transfer mechanisms such as Forster Resonance Energy Transfer (FRET) or Dexter energy transfer among luminophore molecules. This can be particularly useful in downconversion processes where energy absorbed by higher-energy luminophores is non-radiatively transferred to adjacent lower-energy luminophores, which then emit light at longer wavelengths.

Surfactants can solubilise other organic or inorganic luminophores, protecting them from quenching interactions with the solvent or other species in the solution. Therefore, intrinsic luminescent surfactants could enhance quantum dot sensitivity and fine tune their optical properties, as discussed herein below. This can be especially important in aqueous environments where many luminophores are unstable or have low solubility. The self-assembly of surfactant luminophores can lead to a variety of nanostructures with different sizes, shapes, and compositions. These structures can exhibit unique optical properties due to quantum confinement effects, plasmon resonance, or through the control of intermolecular interactions within the assembled structure. By adjusting the surfactant concentration, the type of surfactant, or the conditions of the environment (pH, ionic strength, etc.), it is possible to fine-tune the emission properties of the system for specific downconversion applications. The organised structures formed by surfactant luminophores can also enhance light absorption and scattering, increasing the efficiency of the downconversion process. This can be particularly advantageous in photovoltaic applications where maximising light absorption over a broad range of wavelengths is crucial for improving energy conversion efficiency (https://doi.org/10.1038/s41560-017-0016-9) (https://doi.org/10.1038/nnano.2015.178).

It is thus believed that the luminescent surfactant compounds of the present invention would be used to enhance the conversion efficiency of PV-cells by applying a thin film or multiple thin films to the cell. This will increase the amount of visible light. It may also help to prevent the decomposition of the panels over time.

The application of the luminescent surfactant compounds of the present invention are envisaged to include:
(i) in/as paint and protective coatings; the present invention pertains to novel luminescent organic compounds that function as UV down-converters and exhibit excellent surfactant properties. These compounds are envisioned for application in protective paints, providing enhanced durability, UV protection, and aesthetic benefits. UV radiation poses significant risks to various surfaces, leading to degradation, discolouration, and structural damage. Traditional protective paints often rely on UV absorbers, which can degrade over time, reducing their effectiveness. The luminescent organic compounds in this invention address these limitations by converting harmful UV radiation into less harmful visible light, while also improving the paint's surfactant properties. These compounds exhibit luminescence and surfactant properties thereby capable of converting UV light into visible light, thus providing a dual function of protection and aesthetic enhancement. In addition, due to their surfactant properties, improving the spreadability and adhesion of the paint on various surfaces. The luminescent compounds absorb high-energy UV radiation and re-emit it as lower-energy visible light. This process reduces the amount of UV radiation reaching the substrate, thereby protecting it from UV-induced damage. The surfactant properties can enhance the wetting of surfaces, ensuring uniform paint coverage and improved adhesion. This results in a smoother finish and increased paint longevity. Because of the non-toxic properties of these compounds, they would also be suitable for the use in marine paints and applications;
(ii) in/as sunscreen lotions; the present invention relates to novel luminescent organic compounds that function as UV down-converters, excellent surfactants, and exhibit significantly lower cytotoxicity compared to traditional surfactants like Sodium Dodecyl Sulphate (SDS). These properties make them highly suitable for incorporation into sunscreen lotions, providing effective UV protection, improved formulation stability, and enhanced safety for skin cells. Traditional sunscreens primarily rely on physical or chemical UV filters to protect the skin from harmful ultraviolet radiation. However, some of these filters can cause skin irritation or have limited efficacy. The innovative luminescent organic compounds described in this invention offer a multifaceted approach by converting harmful UV radiation into less harmful visible light, acting as effective surfactants, and being safer for skin application due to their lower cytotoxicity. The compounds can absorb high-energy UV radiation and convert it into lower-energy visible light, providing broad-spectrum UV protection, by covering both UVA and UVB ranges. This process reduces the amount of UV radiation reaching the skin, protecting against UV-induced skin damage. The visible light emission can give the skin a subtle glow, enhancing the cosmetic appeal of the sunscreen lotion. These compounds demonstrated significantly lower cytotoxicity against human keratinocyte (HaCaT) and fibroblast (BJ-5ta) cell lines compared to SDS, ensuring safer application on the skin. The reduced cytotoxicity of these compounds ensures they are gentle on the skin, minimizing the risk of irritation and allergic reactions often associated with traditional sunscreens;
(iii) in plants and insects: incorporating luminescent surfactant compounds of the present invention which facilitate the down-conversion of UV light used for formulating pheromones presents a novel and potentially highly beneficial approach in agricultural practices, particularly in integrated pest management (IPM) strategies. This approach can enhance the efficacy and longevity of pheromone-based products, which are critical for non-toxic pest control.

Several benefits of using luminescent surfactant compounds of the present invention technology include:

### Enhanced UV Protection

(i) Increased Stability of Pheromones: UV radiation can significantly degrade sensitive pheromone molecules, reducing their effectiveness over time. By converting harmful UV light into less damaging wavelengths or into beneficial light, the stability and lifespan of pheromones in the field can be increased;
(ii) Protection of Active Ingredients: Beyond pheromones, other sensitive active ingredients used in agriculture (e.g., certain pesticides or plant growth regulators) could also benefit from enhanced UV protection, preserving their efficacy for longer periods.

### Improved Efficacy of Pheromone-Based Products

(i) Extended-Release Profiles: By stabilizing pheromones against UV degradation, it's possible to achieve more consistent and prolonged release rates. This ensures that the active compounds maintain their integrity and effectiveness, providing continuous pest control without the need for frequent reapplications;
(ii) Enhanced Attractiveness to Target Pests: With the active pheromones remaining effective for longer, the traps or lures will be more attractive to target pests over extended periods. This can lead to more efficient pest management and reduced crop damage.

### Quantum Dots for Bio-Imaging

Amino acid surfactants (AASs) exhibiting NTIL does not fit into one of these classes of NTIL.

AASs are more biodegradable compared to polymers and dendrimers (because these compounds contain multiple branching). The fluorophore of the compound is contained in the surfactant; this is an improvement to quantum dot studies where surfactants are used to encapsulate the active compound containing a fluorophore (Fan et al., 2005). The synthesis and the encapsulation of quantum dots, such as gold nanocrystals or monodisperse Cd/Se are time consuming and expensive (Bruchez Jr et al., 1998). Quantum dots are promising alternatives to organic dyes used in bio imaging. The key to develop efficient quantum dots is to achieve water solubility, and flexible surface chemistry that would couple the fluorescent probes to the targeted material. AASs are easily modified to fit a specific surface chemistry and solubility. In addition hereto, traditional quantum dots are stabilised in solution using surfactants to prevent clustering. Upon activation or use, these surfactant stabilizers are removed and the quantum dots undergo functionalisation.

The Applicant believes that luminescent surfactant compounds of the present invention will not only stabilise existing quantum dots but also will synergistically enhance their optical properties within the solution.

### Optical Sensor Technology

Depending on the tail, head and complexation or chelation ability of the luminescent surfactant compounds of the present invention, the absorption of light and emission can exhibit different emission colours. This can be used as sensors to inter alia detect environmental pollutants or be tuned to absorb specific wavelengths with specific emissions that are sought for applications, i.e., if a certain colour appears it is indicative of a certain metal pollutant, etc.

It has now been surprisingly demonstrated that surfactant compounds having the chemical structure defined and described in terms of the first, second, third and fourth aspects of the present invention exhibit luminescence and furthermore that the use of these luminescent surfactant compounds have superior application in new areas of technology.

As discussed herein above in the background discussion, it is known in the art that certain surfactants may appear to luminesce. However, their ability to luminesce is ascribed either to the addition of a dye or luminescent dopants and not to the surfactant compound's structure.

The Applicant believes that the presently disclosed luminescent surfactant compounds' ability to luminesce is attributed to their molecular structure, which includes chromophores (light-absorbing parts) and fluorophores (light-emitting parts). Here, it is believed that their luminescence is a result of their own electronic transitions. The intrinsic luminescence of these surfactants offers a synergistic relationship with their surfactant properties, leading to enhanced functionalities and applications that are not possible with non-luminescent surfactants or those that require external dopants for luminescence, as are currently employed in the art.

According to the Applicant, the surfactant compounds having the chemical structure defined and described in terms of the first, second, third and fourth aspects of the present invention are distinguished as superior fluorescent surfactants primarily due to their optimal hydrophobic-hydrophilic balance. This balance is achieved through the integration of highly hydrophilic amino acids, which contain amine and carboxylic functional groups, and Guerbet tails which are markedly hydrophobic, attributed to their extensive methylene carbon chains (FIG. 16).

However, the utility of linear surfactants with long carbon chains is constrained by their high pour points, which pose challenges for industrial optimisation. The pour point indicates the lowest temperature at which a surfactant remains fluid, and for long-chain linear surfactants, this parameter is not industrially feasible. Guerbet tails, characterised by their branched structure, effectively reduce the effective carbon chain length (ECCL) while preserving, if not enhancing, their lipophilic properties. This branched configuration mitigates the pour point limitations associated with linear surfactants, thereby facilitating a more favourable balance between hydrophobicity and hydrophilicity; a critical factor in the efficiency of fluorescent surfactants.

This synergy between luminescence ability coupled with the surfactant properties of the disclosed compounds is beneficial in several key areas:

### Simplified System Complexity

The luminescent surfactant compounds of the present invention eliminate the need for external dyes or luminescent dopants, reducing the complexity of the system. This simplicity is advantageous because it avoids potential issues related to the compatibility of the surfactant with the dopant, the stability of the dopant within the surfactant matrix, and the quenching of luminescence that can occur in doped systems. This leads to more straightforward interpretations of experimental results and potentially more robust applications.

### Enhanced Sensitivity and Specificity

The luminescence of especially amino acid-based surfactants of the present invention can be finely tuned to respond to changes in their environment, such as pH, ionic strength, or the presence of specific ions or molecules. This sensitivity is due to the intimate relationship between the surfactant's structure, which dictates its assembly and interaction with its environment, and its luminescent properties. The luminescent surfactant compounds of the present invention thus provide excellent candidates for sensors and probes in chemical, biological, and environmental analyses.

### Real-Time Monitoring of Surfactant Behaviour

The luminescent surfactant compounds of the present invention afford real-time monitoring of their behaviour in solution, including micelle formation, phase transitions, and interactions with other molecules. This is particularly valuable in studying dynamic processes such as self-assembly, where the luminescence can provide immediate feedback on the structural changes occurring within the system. This capability facilitates a deeper understanding of surfactant dynamics and can lead to the development of novel materials and formulations.

### Aggregation-Induced Emission (AIE)

The luminescent surfactant compounds of the present invention exhibit aggregation-induced emission, where their luminescence is enhanced upon aggregation. This property creates a synergistic relationship between the surfactant compounds tendency to self-assemble and its luminescence, as the formation of micelles or other aggregates can significantly increase the emission intensity.

In particular, the Guerbet-type hydrophobic part of the surfactant compound reduces the concentration needed for aggregation (CMC) and therefore enhances the luminescence at lower concentrations. This feature is particularly useful in designing highly sensitive and selective luminescent probes and materials.

### Biocompatibility and Bioimaging Applications

The luminescent surfactant compounds of the present invention that are biocompatible can be directly used for bioimaging applications, where they can serve as probes to visualise cellular structures or track the delivery of drugs encapsulated within surfactant micelles. The synergy between their luminescent properties and their ability to form micellar structures or vesicles is particularly advantageous in this context, as it allows for the simultaneous delivery and tracking of therapeutic agents.

### Photophysical Properties Tuning

The self-assembling nature of surfactants can be leveraged to tune the photophysical properties of the luminescent moieties. For example, the packing of the presently disclosed surfactant molecules in different aggregates can affect the quantum yield, emission wavelength, and photostability of the luminescence. This tunability is a direct result of the interplay between the surfactant's assembly behaviour and its luminescent characteristics.

In summary, the luminescent surfactant compounds of the present invention represent a multifunctional class of materials where the surfactant and luminescent properties are inherently linked, offering enhanced performance and versatility. This synergistic relationship opens up new possibilities in the design of smart materials and sensors, advanced imaging techniques, and novel delivery systems, among other applications.

From the above, it can be seen that new and unexpected applications have been found for luminescent surfactant compounds of the present invention.

The description is presented by way of example only in the cause of providing what is believed to be the most useful and readily understood description of the principles and conceptual aspects of the invention. In this regard, no attempt is made to show structural details of the invention and/or the equipment utilized therein in more detail than is necessary for a fundamental understanding of the invention.

### REFERENCES

Algar, W. R. (2020). Heroes or villains?How nontraditional luminescent materials do and do not enhance bioanalysis and imaging. Chemistry of Materials, 32(12), 4863-4883.
Bruchez Jr, M., Moronne, M., Gin, P., Weiss, S., & Alivisatos, A. P. (1998). Semiconductor nanocrystals as fluorescent biological labels. Science, 281(5385), 2013-2016.
Chen, Y., & Rosenzweig, Z. (2002). Luminescent CdSe quantum dot doped stabilized micelles. Nano letters, 2(11), 1299-1302.
M. Hegazy, A. EI-Tabei, A. Bedair, M. Sadeq, Corrosion Science, 2012, 54, 219-230.
Fan, H., Leve, E. W., Scullin, C., Gabaldon, J., Tallant, D., Bunge, S., Boyle, T., Wilson, M. C., & Brinker, C. J. (2005). Surfactant-assisted synthesis of water-soluble and biocompatible semiconductor quantum dot micelles. Nano Letters, 5(4), 645-648.
Hu, R., Yang, X., Qin, A., & Tang, B. Z. (2021). AIE polymers in sensing, imaging and theranostic applications. Materials Chemistry Frontiers, 5(11), 4073-4088.
Meinardi, F., McDaniel, H., Carulli, F., Colombo, A., Velizhanin, K. A., Makarov, N. S., Simonutti, R., Klimov, V. I. and Brovelli, S., 2015. Highly efficient large-area colourless luminescent solar concentrators using heavy-metal-free colloidal quantum dots. Nature nanotechnology, 10(10), pp. 878-885.
Smith McWilliams, A., Ergülen, S., Ogle, M., de los Reyes, C., Pasquali, M., & Marti, A. (2020). Fluorescent surfactants from common dyes—Rhodamine B and Eosin Y. Pure and Applied Chemistry, 92(2), 265-274.
Tehrani-Bagha, A. R., & Holmberg, K. (2013). Solubilization of hydrophobic dyes in surfactant solutions. Materials, 6(2), 580-608.
Tomalia, D. A., Klajnert-Maculewicz, B., Johnson, K. A. M., Brinkman, H. F., Janaszewska, A. and Hedstrand, D. M., 2019. Non-traditional intrinsic luminescence: inexplicable blue fluorescence observed for dendrimers, macromolecules and small molecular structures lacking traditional/conventional luminophores. Progress in Polymer Science, 90, pp. 35-117.
Traverse, C. J., Pandey, R., Barr, M. C. and Lunt, R. R., 2017. Emergence of highly transparent photovoltaics for distributed applications. Nature Energy, 2(11), pp. 849-860.
Warner, T., Ghiggino, K. P. and Rosengarten, G., 2022. A critical analysis of luminescent solar concentrator terminology and efficiency results. Solar Energy, 246, pp. 119-140.
Zheng, M., Wang, Y., Zhang, D. and Zhu, M., 2023. Fluorescent Materials with Excellent Biocompatibility and Their Application in Bio-Sensing, Bio-Imaging. Biosensors, 13(10), p. 906.
SHARP Corp., 2012. Sharp Develops Solar Cell with World's Highest Conversion Efficiency of 37.7%. https://global.sharp/corporate/news/121205.html [date of access: 17 May 2024].

1-8. (canceled)

9. A composition that is a quantum dot, UV-down converter, solar cell, smart material, or sensor, wherein said composition comprises a luminescent surfactant compound having a chemical structure selected from: 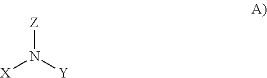
wherein:
(i) X is either COR or CH₂R, wherein R is either R′CH(CH₂)₂R′ or R′CCHCH₂R′, and wherein R′ is CₙH₂ₙ₊₁ (n=integer);
(ii) Y is selected from the group consisting of H; R′; X; (CH₂)ₙCO₂M (n=integer; M=H, R′, Li, Na, K, Rb, Cs, Fr, Er, or Gd); (CH₂)ₙA (n=integer; A=OH, OR′, SH, SR′, NH₂, NHR′, NR′₂, CONH₂, CONHR′, CONR′₂, a mono-aromatic group, a mono-heteroaromatic group, or a halogen); and CH₂CH(CH₃)O(CH₂CH₂O)ₙH (n=integer); and
(iii) Z is selected from the group consisting of H; R′; (CH₂)ₙCO₂M (n=integer; M=H, R′, Li, Na, K, Rb, Cs, Fr, Er, or Gd); CH₂CH(CH₃)O(CH₂CH₂O)ₙH (n=integer); and (CH₂)ₙA (n=integer; A=OH, OR′, SH, SR′, NH₂, NHR′, NR′₂, CONH₂, CONHR′, CONR′₂, a mono-aromatic group, a mono-heteroaromatic group, or a halogen); 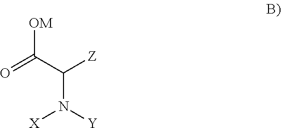
wherein:
(i) X is either COR or CH₂R, wherein R is either R′CH(CH₂)₂R′ or R′CCHCH₂R′, and wherein R′ is CₙH₂ₙ₊₁ (n=integer);
(ii) Y is selected from the group consisting of H; R′; X; (CH₂)ₙCO₂M (n=integer; M=H, R′, Li, Na, K, Rb, Cs, Fr, Er, or Gd); (CH₂)ₙA (n=integer; A=OH, OR′, SH, SR′, NH₂, NHR′, NR′₂, CONH₂, CONHR′, CONR′₂, an aromatic group, a heteroaromatic group, or a halogen); and CH₂CH(CH₃)O(CH₂CH₂O)ₙH (n=integer); and
(iii) Z is selected from the group consisting of H; R′; (CH₂)ₙA (n=integer; A=OH, OR′, SH, SR′, NH₂, NHR′, NR′₂, CONH₂, CONHR′, CONR′₂, an aromatic group, a heteroaromatic group, or a halogen); (CH₂)ₙCO₂M (n=integer; M=H, R′, Li, Na, K, Rb, Cs, Fr, Er, or Gd); 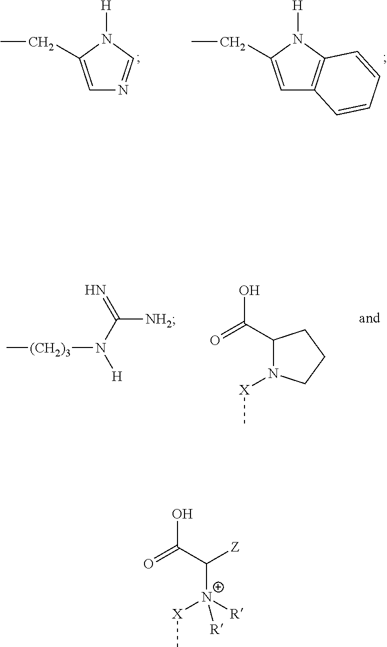
wherein A=OH (SER); SH (CYS); CH₂SMe (MET); COOH (ASP); CH₂COOH (GLU); (CH₂)₃NH₂ (LYS); CONH₂ (ASP); CH₂CONH₂ (GLN); CH(OH)Me (THR); and
wherein ALA=alanine; ASP=aspartic acid; GLU=glutamic acid; GLY=glycine; IDA=iminodiacetic acid; VAL=valine; LEU=leucine; ILE=isoleucine; PHE=phenylalanine; SER=serine; CYS=cysteine; MET=methionine; LYS=lysine; GLN=glutamine; THR=threonine; 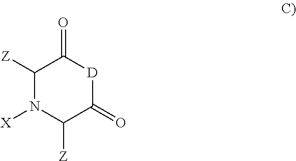
wherein:
(i) X is either COR or CH₂R, wherein R is either R′CH(CH₂)₂R′ or R′CCHCH₂R′, and wherein R′ is CₙH₂ₙ₊₁ (n=integer);
(ii) D is selected from the group consisting of O; N(CH₂)ₙA and N(C₆H₄)ₙA (n=integer; A=OH, OR′, SH, SR′, NH₂, NHR′, NR′₂, CONH₂, CONHR′, CONR′₂, an aromatic group, a heteroaromatic group or a halogen); and
(iii) Z is selected from the group consisting of H; R′; N(CH₂)ₙA (n=integer; A=OH, OR′, SH, SR′, NH₂, NHR′, NR′₂, CONH₂, CONHR′, CONR′₂, an aromatic group, a heteroaromatic group, or a halogen); and (CH₂)ₙCO₂M (n=integer; M=H, R′, Li, Na, K, Rb, Cs, Fr, Er, or Gd); and 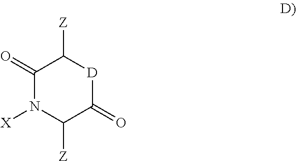
wherein:
(i) X is either COR or CH₂R, wherein R is either R′CH(CH₂)₂R′ or R′CCHCH₂R′, and wherein R′ is CₙH₂ₙ₊₁ (n=integer);
(ii) D is selected from the group consisting of O; N(CH₂)ₙA and N(C₆H₄)ₙA (n=integer; A=OH, OR′, SH, SR′, NH₂, NHR′, NR′₂, CONH₂, CONHR′, CONR′₂, an aromatic group, a heteroaromatic group or a halogen); and
(iii) Z is selected from the group consisting of H; R′; N(CH₂)ₙA (n=integer; A=OH, OR′, SH, SR′, NH₂, NHR′, NR′₂, CONH₂, CONHR′, CONR′2, an aromatic group, a heteroaromatic group, or a halogen); and (CH₂)ₙCO₂M (n=integer; M=H, R′, Li, Na, K, Rb, Cs, Fr, Er, or Gd).

10. The composition according to claim 9, wherein the luminescent surfactant compound has the following chemical structure: 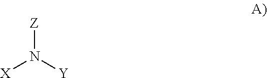
wherein:
(i) X is either COR or CH₂R, wherein R is either R′CH(CH₂)₂R′ or R′CCHCH₂R′, and wherein R′ is CₙH₂ₙ₊₁ (n=integer);
(ii) Y is selected from the group consisting of H; R′; X; (CH₂)CO₂M (n=integer; M=H, R′, Li, Na, K, Rb, Cs, Fr, Er, or Gd); (CH₂)ₙA (n=integer; A=OH, OR′, SH, SR′, NH₂, NHR′, NR′₂, CONH₂, CONHR′, CONR′₂, a mono-aromatic group, a mono-heteroaromatic group, or a halogen); and CH₂CH(CH₃)O(CH₂CH₂O)ₙH (n=integer); and
(iii) Z is selected from the group consisting of H; R′; (CH₂)ₙCO₂M (n=integer; M=H, R′, Li, Na, K, Rb, Cs, Fr, Er, or Gd); CH₂CH(CH₃)O(CH₂CH₂O)ₙH (n=integer); and (CH₂)ₙA (n=integer; A=OH, OR′, SH, SR′, NH₂, NHR′, NR′₂, CONH₂, CONHR′, CONR′₂, a mono-aromatic group, a mono-heteroaromatic group, or a halogen).

11. The composition according to claim 9, wherein the luminescent surfactant compound has the following chemical structure: 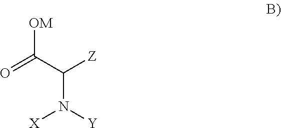
wherein:
(i) X is either COR or CH₂R, wherein R is either R′CH(CH₂)₂R′ or R′CCHCH₂R′, and wherein R′ is CₙH₂n+1 (n=integer);
(ii) Y is selected from the group consisting of H; R′; X; (CH₂)ₙCO₂M (n=integer; M=H, R′, Li, Na, K, Rb, Cs, Fr, Er, or Gd); (CH₂)ₙA (n=integer; A=OH, OR′, SH, SR′, NH₂, NHR′, NR′₂, CONH₂, CONHR′, CONR′₂, an aromatic group, a heteroaromatic group, or a halogen); and CH₂CH(CH₃)O(CH₂CH₂O)ₙH (n=integer); and
(iii) Z is selected from the group consisting of H; R′; (CH₂)ₙA (n=integer; A=OH, OR′, SH, SR′, NH₂, NHR′, NR′₂, CONH₂, CONHR′, CONR′₂, an aromatic group, a heteroaromatic group, or a halogen); (CH₂)ₙCO₂M (n=integer; M=H, R′, Li, Na, K, Rb, Cs, Fr, Er, or Gd); 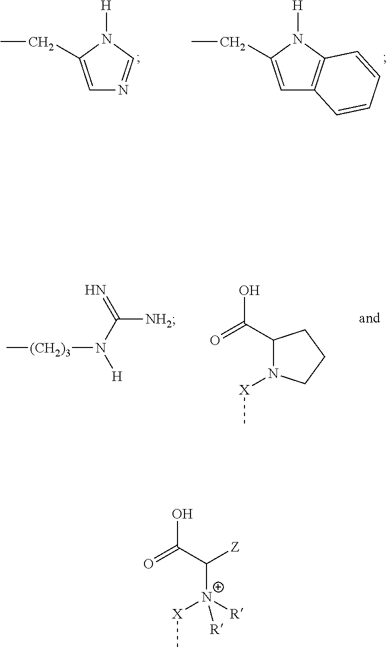
wherein A=OH (SER); SH (CYS); CH₂SMe (MET); COOH (ASP); CH₂COOH (GLU); (CH₂)₃NH₂ (LYS); CONH₂ (ASP); CH₂CONH₂ (GLN); CH(OH)Me (THR); and
wherein ALA=alanine; ASP=aspartic acid; GLU=glutamic acid; GLY=glycine; IDA=iminodiacetic acid; VAL=valine; LEU=leucine; ILE=isoleucine; PHE=phenylalanine; SER=serine; CYS=cysteine; MET=methionine; LYS=lysine; GLN=glutamine; THR=threonine.

12. The composition according to claim 9, wherein the luminescent surfactant compound has the following chemical structure: 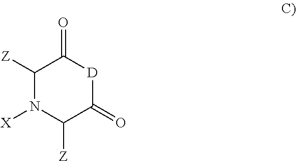
wherein:
(i) X is either COR or CH₂R, wherein R is either R′CH(CH₂)₂R′ or R′CCHCH₂R′, and wherein R′ is CₙH₂ₙ₊₁ (n=integer);
(ii) D is selected from the group consisting of O; N(CH₂)ₙA and N(C₆H₄)ₙA (n=integer; A=OH, OR′, SH, SR′, NH₂, NHR′, NR′₂, CONH₂, CONHR′, CONR′₂, an aromatic group, a heteroaromatic group or a halogen); and
(iii) Z is selected from the group consisting of H; R′; N(CH₂)ₙA (n=integer; A=OH, OR′, SH, SR′, NH₂, NHR′, NR′2, CONH₂, CONHR′, CONR′₂, an aromatic group, a heteroaromatic group, or a halogen); and (CH₂)ₙCO₂M (n=integer; M=H, R′, Li, Na, K, Rb, Cs, Fr, Er, or Gd).

13. The composition according to claim 9, wherein the luminescent surfactant compound has the following chemical structure: 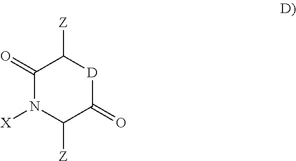
wherein:
(i) X is either COR or CH₂R, wherein R is either R′CH(CH₂)₂R′ or R′CCHCH₂R′, and wherein R′ is CₙH₂ₙ₊₁ (n=integer);
(ii) D is selected from the group consisting of O; N(CH₂)ₙA and N(C₆H₄)ₙA (n=integer; A=OH, OR′, SH, SR′, NH₂, NHR′, NR′2, CONH₂, CONHR′, CONR′₂, an aromatic group, a heteroaromatic group or a halogen); and
(iii) Z is selected from the group consisting of H; R′; N(CH₂)ₙA (n=integer; A=OH, OR′, SH, SR′, NH₂, NHR′, NR′₂, CONH₂, CONHR′, CONR′₂, an aromatic group, a heteroaromatic group, or a halogen); and (CH₂)ₙCO₂M (n=integer; M=H, R′, Li, Na, K, Rb, Cs, Fr, Er, or Gd).

14. A method for manufacturing a UV-down converter, solar cell, smart material, or sensor wherein said method comprises use of a luminescent linear heteroatom functionalized surfactant compound.

15. The method according to claim 14, wherein the luminescent linear heteroatom functionalized surfactant compound comprises both saturated and unsaturated hydrocarbons.

16. The method according to claim 14, wherein the luminescent linear heteroatom functionalized surfactant compound is a commercially-available surfactant.

17. The method according to claim 14, wherein the luminescent linear heteroatom functionalized surfactant compound is selected from Rewoteric AMC 2CNM; Rewopol SBF A30B; Tego SMS 60; Varisoft 432 CG and Varisoft 300.

18. The method according to claim 14, wherein the luminescent linear heteroatom functionalized surfactant compound has a chemical structure selected from: 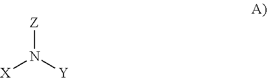
wherein:
(i) X is either COR or CH₂R, wherein R is either R′CH(CH₂)₂R′ or R′CCHCH₂R′, and wherein R′ is CH₂ₙ₊₁ (n=integer);
(ii) Y is selected from the group consisting of H; R′; X; (CH₂)ₙCO₂M (n=integer; M=H, R′, Li, Na, K, Rb, Cs, Fr, Er, or Gd); (CH₂)ₙA (n=integer; A=OH, OR′, SH, SR′, NH₂, NHR′, NR′₂, CONH₂, CONHR′, CONR′₂, a mono-aromatic group, a mono-heteroaromatic group, or a halogen); and CH₂CH(CH₃)O(CH₂CH₂O)ₙH (n=integer); and
(iii) Z is selected from the group consisting of H; R′; (CH₂)ₙCO₂M (n=integer; M=H, R′, Li, Na, K, Rb, Cs, Fr, Er, or Gd); CH₂CH(CH₃)O(CH₂CH₂O)ₙH (n=integer); and (CH₂)ₙA (n=integer; A=OH, OR′, SH, SR′, NH₂, NHR′, NR′₂, CONH₂, CONHR′, CONR′₂, a mono-aromatic group, a mono-heteroaromatic group, or a halogen); 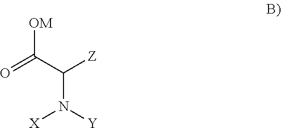
wherein:
(i) X is either COR or CH₂R, wherein R is either R′CH(CH₂)₂R′ or R′CCHCH₂R′, and wherein R′ is CₙH₂ₙ₊₁ (n=integer);
(ii) Y is selected from the group consisting of H; R′; X; (CH₂)ₙCO₂M (n=integer; M=H, R′, Li, Na, K, Rb, Cs, Fr, Er, or Gd); (CH₂)ₙA (n=integer; A=OH, OR′, SH, SR′, NH₂, NHR′, NR′₂, CONH₂, CONHR′, CONR′2, an aromatic group, a heteroaromatic group, or a halogen); and CH₂CH(CH₃)O(CH₂CH₂O)ₙH (n=integer); and
(iii) Z is selected from the group consisting of H; R′; (CH₂)ₙA (n=integer; A=OH, OR′, SH, SR′, NH₂, NHR′, NR′₂, CONH₂, CONHR′, CONR′₂, an aromatic group, a heteroaromatic group, or a halogen); (CH₂)ₙCO₂M (n=integer; M=H, R′, Li, Na, K, Rb, Cs, Fr, Er, or Gd); 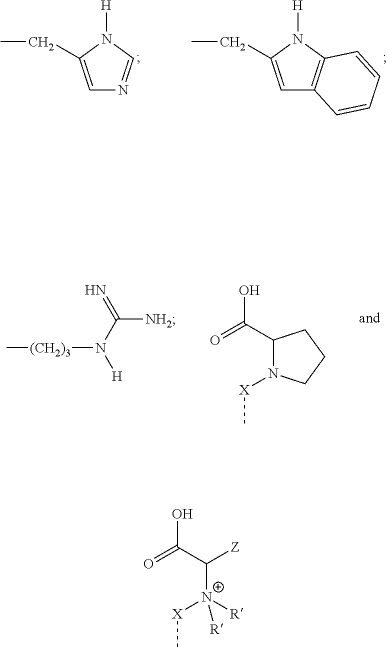
wherein A=OH (SER); SH (CYS); CH₂SMe (MET); COOH (ASP); CH₂COOH (GLU); (CH₂)₃NH₂ (LYS); CONH₂ (ASP); CH₂CONH₂ (GLN); CH(OH)Me (THR); and
wherein ALA=alanine; ASP=aspartic acid; GLU=glutamic acid; GLY=glycine; IDA=iminodiacetic acid; VAL=valine; LEU=leucine; ILE=isoleucine; PHE=phenylalanine; SER=serine; CYS=cysteine; MET=methionine; LYS=lysine; GLN=glutamine; THR=threonine; 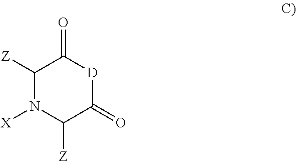
wherein:
(i) X is either COR or CH₂R, wherein R is either R′CH(CH₂)₂R′ or R′CCHCH₂R′, and wherein R′ is CH₂ₙ₊₁ (n=integer);
(ii) D is selected from the group consisting of O; N(CH₂)ₙA and N(C₆H₄)ₙA (n=integer; A=OH, OR′, SH, SR′, NH₂, NHR′, NR′₂, CONH₂, CONHR′, CONR′₂, an aromatic group, a heteroaromatic group or a halogen); and
(iii) Z is selected from the group consisting of H; R′; N(CH₂)ₙA (n=integer; A=OH, OR′, SH, SR′, NH₂, NHR′, NR′₂, CONH₂, CONHR′, CONR′₂, an aromatic group, a heteroaromatic group, or a halogen); and (CH₂)ₙCO₂M (n=integer; M=H, R′, Li, Na, K, Rb, Cs, Fr, Er, or Gd); and 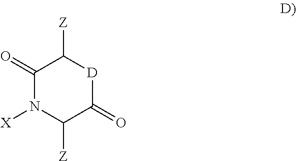
wherein:
(i) X is either COR or CH₂R, wherein R is either R′CH(CH₂)₂R′ or R′CCHCH₂R′, and wherein R′ is CₙH₂ₙ₊₁ (n=integer);
(ii) D is selected from the group consisting of O; N(CH₂)ₙA and N(C₆H₄)ₙA (n=integer; A=OH, OR′, SH, SR′, NH₂, NHR′, NR′₂, CONH₂, CONHR′, CONR′₂, an aromatic group, a heteroaromatic group or a halogen); and
(iii) Z is selected from the group consisting of H; R′; N(CH₂)ₙA (n=integer; A=OH, OR′, SH, SR′, NH₂, NHR′, NR′2, CONH₂, CONHR′, CONR′₂, an aromatic group, a heteroaromatic group, or a halogen); and (CH₂)ₙCO₂M (n=integer; M=H, R′, Li, Na, K, Rb, Cs, Fr, Er, or Gd).

19. The method according to claim 14, wherein the luminescent linear heteroatom functionalized surfactant compound has a chemical structure selected from: 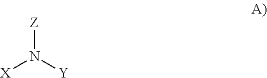
wherein:
(i) X is either COR or CH₂R, wherein R is either R′CH(CH₂)₂R′ or R′CCHCH₂R′, and wherein R′ is CₙH₂n+1 (n=integer);
(ii) Y is selected from the group consisting of H; R′; X; (CH₂)ₙCO₂M (n=integer; M=H, R′, Li, Na, K, Rb, Cs, Fr, Er, or Gd); (CH₂)ₙA (n=integer; A=OH, OR′, SH, SR′, NH₂, NHR′, NR′2, CONH₂, CONHR′, CONR′₂, a mono-aromatic group, a mono-heteroaromatic group, or a halogen); and CH₂CH(CH₃)O(CH₂CH₂O)ₙH (n=integer); and
(iii) Z is selected from the group consisting of H; R′; (CH₂)ₙCO₂M (n=integer; M=H, R′, Li, Na, K, Rb, Cs, Fr, Er, or Gd); CH₂CH(CH₃)O(CH₂CH₂O)ₙH (n=integer); and (CH₂)ₙA (n=integer; A=OH, OR′, SH, SR′, NH₂, NHR′, NR′₂, CONH₂, CONHR′, CONR′₂, a mono-aromatic group, a mono-heteroaromatic group, or a halogen).

20. The method according to claim 14, wherein the luminescent linear heteroatom functionalized surfactant compound has a chemical structure selected from: 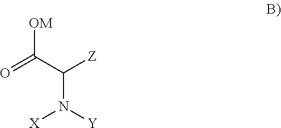
wherein:
(i) X is either COR or CH₂R, wherein R is either R′CH(CH₂)₂R′ or R′CCHCH₂R′, and wherein R′ is CₙH₂ₙ₊₁ (n=integer);
(ii) Y is selected from the group consisting of H; R′; X; (CH₂)ₙCO₂M (n=integer; M=H, R′, Li, Na, K, Rb, Cs, Fr, Er, or Gd); (CH₂)ₙA (n=integer; A=OH, OR′, SH, SR′, NH₂, NHR′, NR′₂, CONH₂, CONHR′, CONR′2, an aromatic group, a heteroaromatic group, or a halogen); and CH₂CH(CH₃)O(CH₂CH₂O)ₙH (n=integer); and
(iii) Z is selected from the group consisting of H; R′; (CH₂)ₙA (n=integer; A=OH, OR′, SH, SR′, NH₂, NHR′, NR′₂, CONH₂, CONHR′, CONR′₂, an aromatic group, a heteroaromatic group, or a halogen); (CH₂)ₙCO₂M (n=integer; M=H, R′, Li, Na, K, Rb, Cs, Fr, Er, or Gd); 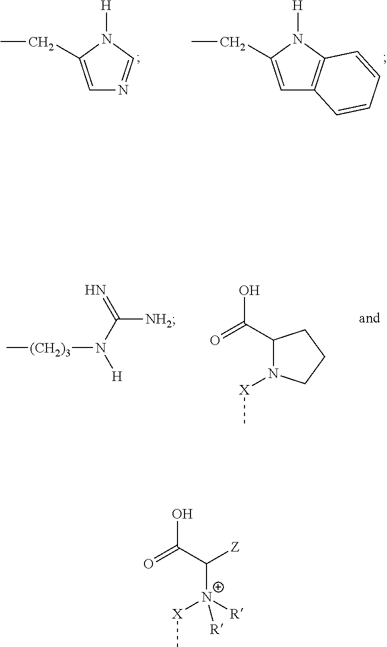
wherein A=OH (SER); SH (CYS); CH₂SMe (MET); COOH (ASP); CH₂COOH (GLU); (CH₂)₃NH₂ (LYS); CONH₂ (ASP); CH₂CONH₂ (GLN); CH(OH)Me (THR); and
wherein ALA=alanine; ASP=aspartic acid; GLU=glutamic acid; GLY=glycine; IDA=iminodiacetic acid; VAL=valine; LEU=leucine; ILE=isoleucine; PHE=phenylalanine; SER=serine; CYS=cysteine; MET=methionine; LYS=lysine; GLN=glutamine; THR=threonine.

21. The method according to claim 14, wherein the luminescent linear heteroatom functionalized surfactant compound has a chemical structure selected from: 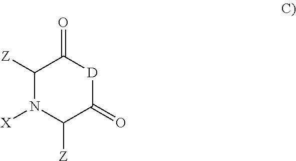
wherein:
(i) X is either COR or CH₂R, wherein R is either R′CH(CH₂)₂R′ or R′CCHCH₂R′, and wherein R′ is CₙH₂ₙ₊₁ (n=integer);
(ii) D is selected from the group consisting of O; N(CH₂)ₙA and N(C₆H₄)ₙA (n=integer; A=OH, OR′, SH, SR′, NH₂, NHR′, NR′₂, CONH₂, CONHR′, CONR′₂, an aromatic group, a heteroaromatic group or a halogen); and
(iii) Z is selected from the group consisting of H; R′; N(CH₂)ₙA (n=integer; A=OH, OR′, SH, SR′, NH₂, NHR′, NR′₂, CONH₂, CONHR′, CONR′₂, an aromatic group, a heteroaromatic group, or a halogen); and (CH₂)ₙCO₂M (n=integer; M=H, R′, Li, Na, K, Rb, Cs, Fr, Er, or Gd).

22. The method according to claim 14, wherein the luminescent linear heteroatom functionalized surfactant compound has a chemical structure selected from: 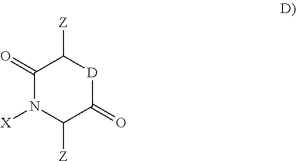
wherein:
(i) X is either COR or CH₂R, wherein R is either R′CH(CH₂)₂R′ or R′CCHCH₂R′, and wherein R′ is CₙH₂ₙ₊₁ (n=integer);
(ii) D is selected from the group consisting of O; N(CH₂)ₙA and N(C₆H₄)ₙA (n=integer; A=OH, OR′, SH, SR′, NH₂, NHR′, NR′₂, CONH₂, CONHR′, CONR′₂, an aromatic group, a heteroaromatic group or a halogen); and
(iii) Z is selected from the group consisting of H; R′; N(CH₂)ₙA (n=integer; A=OH, OR′, SH, SR′, NH₂, NHR′, NR′2, CONH₂, CONHR′, CONR′₂, an aromatic group, a heteroaromatic group, or a halogen); and (CH₂)ₙCO₂M (n=integer; M=H, R′, Li, Na, K, Rb, Cs, Fr, Er, or Gd).

23. A method for bio-imagining wherein said method comprises the use of a luminescent surfactant compound having a chemical structure selected from: 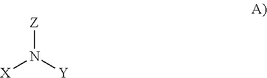
wherein:
(i) X is either COR or CH₂R, wherein R is either R′CH(CH₂)₂R′ or R′CCHCH₂R′, and wherein R′ is CH₂ₙ₊₁ (n=integer);
(ii) Y is selected from the group consisting of H; R′; X; (CH₂)ₙCO₂M (n=integer; M=H, R′, Li, Na, K, Rb, Cs, Fr, Er, or Gd); (CH₂)ₙA (n=integer; A=OH, OR′, SH, SR′, NH₂, NHR′, NR′₂, CONH₂, CONHR′, CONR′₂, a mono-aromatic group, a mono-heteroaromatic group, or a halogen); and CH₂CH(CH₃)O(CH₂CH₂O)ₙH (n=integer); and
(iii) Z is selected from the group consisting of H; R′; (CH₂)ₙCO₂M (n=integer; M=H, R′, Li, Na, K, Rb, Cs, Fr, Er, or Gd); CH₂CH(CH₃)O(CH₂CH₂O)ₙH (n=integer); and (CH₂)ₙA (n=integer; A=OH, OR′, SH, SR′, NH₂, NHR′, NR′2, CONH₂, CONHR′, CONR′2, a mono-aromatic group, a mono-heteroaromatic group, or a halogen); 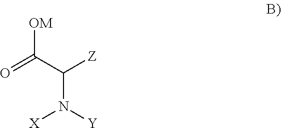
wherein:
(i) X is either COR or CH₂R, wherein R is either R′CH(CH₂)₂R′ or R′CCHCH₂R′, and wherein R′ is CₙH₂n+1 (n=integer);
(ii) Y is selected from the group consisting of H; R′; X; (CH₂)ₙCO₂M (n=integer; M=H, R′, Li, Na, K, Rb, Cs, Fr, Er, or Gd); (CH₂)ₙA (n=integer; A=OH, OR′, SH, SR′, NH₂, NHR′, NR′₂, CONH₂, CONHR′, CONR′₂, an aromatic group, a heteroaromatic group, or a halogen); and CH₂CH(CH₃)O(CH₂CH₂O)ₙH (n=integer); and
(iii) Z is selected from the group consisting of H; R′; (CH₂)ₙA (n=integer; A=OH, OR′, SH, SR′, NH₂, NHR′, NR′₂, CONH₂, CONHR′, CONR′₂, an aromatic group, a heteroaromatic group, or a halogen); (CH₂)ₙCO₂M (n=integer; M=H, R′, Li, Na, K, Rb, Cs, Fr, Er, or Gd); 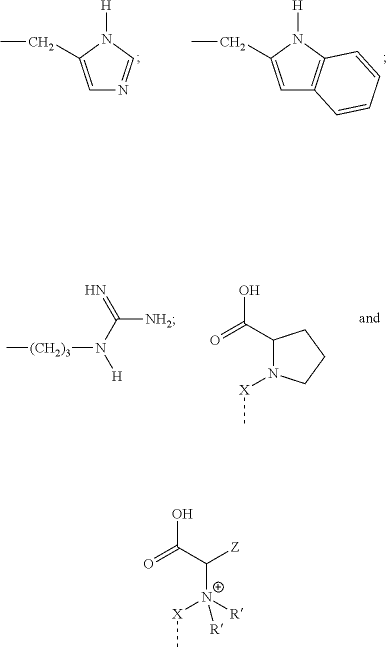
wherein A=OH (SER); SH (CYS); CH₂SMe (MET); COOH (ASP); CH₂COOH (GLU); (CH₂)₃NH₂ (LYS); CONH₂ (ASP); CH₂CONH₂ (GLN); CH(OH)Me (THR); and
wherein ALA=alanine; ASP=aspartic acid; GLU=glutamic acid; GLY=glycine; IDA=iminodiacetic acid; VAL=valine; LEU=leucine; ILE=isoleucine; PHE=phenylalanine; SER=serine; CYS=cysteine; MET=methionine; LYS=lysine; GLN=glutamine; THR=threonine; 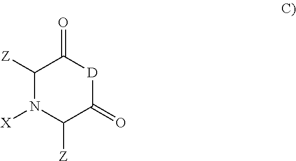
wherein:
(i) X is either COR or CH₂R, wherein R is either R′CH(CH₂)₂R′ or R′CCHCH₂R′, and wherein R′ is CₙH₂ₙ₊₁ (n=integer);
(ii) D is selected from the group consisting of O; N(CH₂)ₙA and N(C₆H₄)ₙA (n=integer; A=OH, OR′, SH, SR′, NH₂, NHR′, NR′2, CONH₂, CONHR′, CONR′₂, an aromatic group, a heteroaromatic group or a halogen); and
(iii) Z is selected from the group consisting of H; R′; N(CH₂)ₙA (n=integer; A=OH, OR′, SH, SR′, NH₂, NHR′, NR′₂, CONH₂, CONHR′, CONR′₂, an aromatic group, a heteroaromatic group, or a halogen); and (CH₂)ₙCO₂M (n=integer; M=H, R′, Li, Na, K, Rb, Cs, Fr, Er, or Gd); and 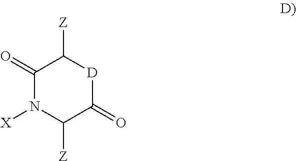
wherein:
(i) X is either COR or CH₂R, wherein R is either R′CH(CH₂)₂R′ or R′CCHCH₂R′, and wherein R′ is CₙH₂ₙ₊₁ (n=integer);
(ii) D is selected from the group consisting of O; N(CH₂)ₙA and N(C₆H₄)ₙA (n=integer; A=OH, OR′, SH, SR′, NH₂, NHR′, NR′2, CONH₂, CONHR′, CONR′₂, an aromatic group, a heteroaromatic group or a halogen); and
(iii) Z is selected from the group consisting of H; R′; N(CH₂)ₙA (n=integer; A=OH, OR′, SH, SR′, NH₂, NHR′, NR′₂, CONH₂, CONHR′, CONR′2, an aromatic group, a heteroaromatic group, or a halogen); and (CH₂)ₙCO₂M (n=integer; M=H, R′, Li, Na, K, Rb, Cs, Fr, Er, or Gd).

24. The method for bio-imagining according to claim 23, wherein said method comprises the use of the luminescent surfactant compound having the following chemical structure: 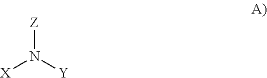
wherein:
(i) X is either COR or CH₂R, wherein R is either R′CH(CH₂)₂R′ or R′CCHCH₂R′, and wherein R′ is CₙH₂ₙ₊₁ (n=integer);
(ii) Y is selected from the group consisting of H; R′; X; (CH₂)ₙCO₂M (n=integer; M=H, R′, Li, Na, K, Rb, Cs, Fr, Er, or Gd); (CH₂)ₙA (n=integer; A=OH, OR′, SH, SR′, NH₂, NHR′, NR′2, CONH₂, CONHR′, CONR′2, a mono-aromatic group, a mono-heteroaromatic group, or a halogen); and CH₂CH(CH₃)O(CH₂CH₂O)~H (n=integer); and
(iii) Z is selected from the group consisting of H; R′; (CH₂)ₙCO₂M (n=integer; M=H, R′, Li, Na, K, Rb, Cs, Fr, Er, or Gd); CH₂CH(CH₃)O(CH₂CH₂O)ₙH (n=integer); and (CH₂)ₙA (n=integer; A=OH, OR′, SH, SR′, NH₂, NHR′, NR′₂, CONH₂, CONHR′, CONR′₂, a mono-aromatic group, a mono-heteroaromatic group, or a halogen).

25. The method for bio-imagining according to claim 23, wherein said method comprises the use of the luminescent surfactant compound having the following chemical structure: 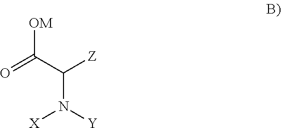
wherein:
(i) X is either COR or CH₂R, wherein R is either R′CH(CH₂)₂R′ or R′CCHCH₂R′, and wherein R′ is CₙH₂ₙ₊₁ (n=integer);
(ii) Y is selected from the group consisting of H; R′; X; (CH₂)ₙCO₂M (n=integer; M=H, R′, Li, Na, K, Rb, Cs, Fr, Er, or Gd); (CH₂)ₙA (n=integer; A=OH, OR′, SH, SR′, NH₂, NHR′, NR′₂, CONH₂, CONHR′, CONR′₂, an aromatic group, a heteroaromatic group, or a halogen); and CH₂CH(CH₃)O(CH₂CH₂O)ₙH (n=integer); and
(iii) Z is selected from the group consisting of H; R′; (CH₂)ₙA (n=integer; A=OH, OR′, SH, SR′, NH₂, NHR′, NR′₂, CONH₂, CONHR′, CONR′₂, an aromatic group, a heteroaromatic group, or a halogen); (CH₂)ₙCO₂M (n=integer; M=H, R′, Li, Na, K, Rb, Cs, Fr, Er, or Gd); 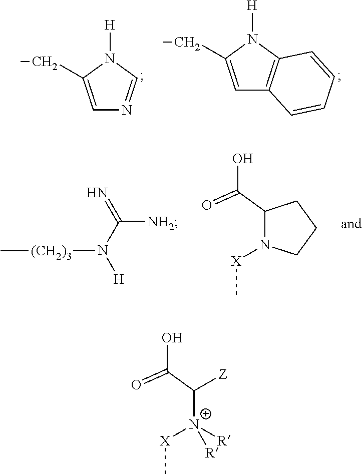
wherein A=OH (SER); SH (CYS); CH₂SMe (MET); COOH (ASP); CH₂COOH (GLU); (CH₂)₃NH₂ (LYS); CONH₂ (ASP); CH₂CONH₂ (GLN); CH(OH)Me (THR); and
wherein ALA=alanine; ASP=aspartic acid; GLU=glutamic acid; GLY=glycine; IDA=iminodiacetic acid; VAL=valine; LEU=leucine; ILE=isoleucine; PHE=phenylalanine; SER=serine; CYS=cysteine; MET=methionine; LYS=lysine; GLN=glutamine; THR=threonine.

26. The method for bio-imagining according to claim 23, wherein said method comprises the use of the luminescent surfactant compound having the following chemical structure: 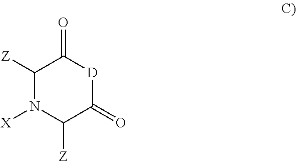
wherein:
(i) X is either COR or CH₂R, wherein R is either R′CH(CH₂)₂R′ or R′CCHCH₂R′, and wherein R′ is CₙH₂ₙ₊₁ (n=integer);
(ii) D is selected from the group consisting of O; N(CH₂)ₙA and N(C₆H₄)ₙA (n=integer; A=OH, OR′, SH, SR′, NH₂, NHR′, NR′₂, CONH₂, CONHR′, CONR′₂, an aromatic group, a heteroaromatic group or a halogen); and
(iii) Z is selected from the group consisting of H; R′; N(CH₂)ₙA (n=integer; A=OH, OR′, SH, SR′, NH₂, NHR′, NR′₂, CONH₂, CONHR′, CONR′₂, an aromatic group, a heteroaromatic group, or a halogen); and (CH₂)ₙCO₂M (n=integer; M=H, R′, Li, Na, K, Rb, Cs, Fr, Er, or Gd).

27. The method for bio-imagining according to claim 23, wherein said method comprises the use of the luminescent surfactant compound having the following chemical structure: 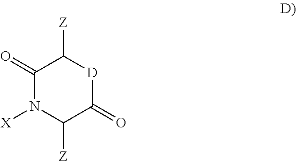
wherein:
(i) X is either COR or CH₂R, wherein R is either R′CH(CH₂)₂R′ or R′CCHCH₂R′, and wherein R′ is CₙH₂ₙ₊₁ (n=integer);
(ii) D is selected from the group consisting of O; N(CH₂)ₙA and N(C₆H₄)ₙA (n=integer; A=OH, OR′, SH, SR′, NH₂, NHR′, NR′2, CONH₂, CONHR′, CONR′₂, an aromatic group, a heteroaromatic group or a halogen); and
(iii) Z is selected from the group consisting of H; R′; N(CH₂)ₙA (n=integer; A=OH, OR′, SH, SR′, NH₂, NHR′, NR′₂, CONH₂, CONHR′, CONR′₂, an aromatic group, a heteroaromatic group, or a halogen); and (CH₂)ₙCO₂M (n=integer; M=H, R′, Li, Na, K, Rb, Cs, Fr, Er, or Gd).
